(19) United States
(12) Patent Application Publication
(10) Pub. No.: US 2016/0228140 A1
(43) Pub. Date: Aug. 11, 2016

(54) DEVICE, KIT AND METHOD FOR PROLONGED LIFTING OF A TISSUE DURING ENDOSCOPIC PROCEDURE

(71) Applicant: VICUT MEDICAL DEVICES LTD., Alonei Abba (IL)

(72) Inventors: Adi Strauss, Alonei Abba (IL); Victor Levin, Haifa (IL); Tali Strauss, Alonei Abba (IL); Refael Amit Cohen, Tel-Aviv-Yafo (IL); Amram Efrat, Tel Aviv (IL)

(73) Assignee: Vicut Medical Devices Ltd.

(21) Appl. No.: 14/392,357

(22) PCT Filed: Jun. 25, 2014
(86) PCT No.: PCT/IL2014/050569

§ 371 Date: Dec. 26, 2015

Related U.S. Application Data
(60) Provisional application No. 61/839,411, filed on Jun. 26, 2013.
(60) Provisional application No. 61/839,399, filed on Jun. 26, 2013.

(51) Int. Cl. A61B 17/3203

(52) U.S. Cl. CPC A61B 17/3203; A61B 2017/320044

(57) ABSTRACT

Provided is a method for lifting a first tissue layer with respect to a second tissue layer adjacent thereto, during an endoscopic procedure. The method includes delivering a composition configured to undergo phase transition, to a region between the first tissue layer and the second tissue layer, and controllably inducing phase transition of the composition from a liquid state to a solid state thereof. Further provided is a device and a kit for lifting a first tissue layer with respect to a second tissue layer adjacent thereto, during an endoscopic procedure. The device includes an injection module having an elongate body, a proximal region and a distal region. The distal region may include at least one outlet to a region between the first tissue layer and the second tissue layer, and a phase transition module.

### FIELD

The present invention is related to devices and methods for separating layers of a target tissue by delivering a composition, configured to undergo phase transition, to the target tissue and by inducing the phase transition of the composition. The invention further relates to an endoscopic system, comprising such devices. The present invention further encompasses the uses of the devices and methods, for example, in endomucosal resection (EMR) and endoscopic submucosal dissection (ESD).

### BACKGROUND

A sessile lesion is a broad-based lesion without a clear stalk. There exist many sessile lesions types which pose a high risk of malignancy. The following table presents a classification of different cases of the sessile lesions which lead to cancer.

TABLE 1
Cancerous sessile lesions

| Type cancer | | | |
|---|---|---|---|
| caused by | Percent of | Area or method | |
| sessile lesions | cases/yr | of treatment | Notes |
| Skin cancer | 4.2 | Skin cancer | In some cases it is hard |
| | | generally | to remove the lesion since |
| | | develops in the | it is depressed and very |
| | | epidermis | large |
| Pancreatic | 13 | Laparoscopy | High rate of mortality - a |
| cancer | | | shortage of methods for |
| | | | effective treatment |
| Liver cancer | 1.1 | Laparoscopy | Including pediatric |
| Female | 10 | Laparoscopy | Includes cervix, tube, |
| reproductive | | | endometrial sessile lesions |
| cancers | | | |
| Lymph node | 1.2 | Laparoscopy | Other way of treatment is |
| cancer | | | chemotherapy |
| GI tract cancer | 4 | GI tract | High rate of mortality - a |
| | | endoscopy | shortage of methods for |
| | | | effective treatment |
| Lung cancer | 20 | Laparoscopy | High rate of mortality - a |
| | | | shortage of methods for |
| | | | effective treatment |
| Appendicitis | 22 | Laparoscopy | One of the most common |
| | | | minimal invasive surgeries |

Current methods of treatment of sessile lesions are defined under the term of minimally invasive surgeries. Sessile lesions are a challenging case for minimally invasive surgery, as there is a critical shortage of methods and instruments to improve procedure yields and there is a major challenge to prepare the lesion and to remove it.

Laparoscopy surgery is a modern surgical technique in which operations in the abdomen are performed through small incisions (usually 0.5-1.5 cm) as compared to the larger incisions needed in laparotomy. Keyhole surgery is assisted by displaying magnified images of surgical elements on suitable monitors. Laparoscopic surgery includes operations within the abdominal or pelvic cavities, whereas keyhole surgery performed on the thoracic or chest cavity is called thoracoscopic surgery. Laparoscopic and thoracoscopic surgery belong to the broader field of endoscopy.

There are a number of advantages to the patient undergoing the laparoscopic surgery as compared to open surgeries. These include reduced pain due to smaller incisions and hemorrhaging, and shorter recovery time.

The key element in laparoscopic surgery is the use of the laparoscope. Two types of laparoscopes are commonly used. The first one includes telescopic rod lens system that is usually connected to a video camera placed on the end of the endoscope. The second one is a digital laparoscope where the charge-coupled device is placed at the end of the laparoscope, eliminating the rod lens system. Said laparoscope further includes a fiber optic cable system connected to a ‘cold’ light source (halogen or xenon), to illuminate the operative field, inserted through a 5 mm or 10 mm cannula and/or trocar to view the operative field. The abdomen is usually insufflated, or essentially blown up like a balloon, with carbon dioxide gas. This elevates the abdominal wall above the internal organs like a dome to create a working and viewing space. Carbon dioxide gas is used because it is common to the human body and can be absorbed by tissue and removed by the respiratory system. It is also non-flammable, which is important because electrosurgical devices are commonly used in laparoscopic procedures.

Endoscopic mucosal (or mucosectomy) resection (EMR) is a technique used to remove cancerous or other abnormal lesions found in the digestive tract. Mucosectomy is a partial-thickness resection of the bowel wall. The resection plane is in the deep submucosa at the junction to the muscularis propriety. Mucosectomy was originally developed for obtaining a larger biopsy specimen then called “strip biopsy”, but evolved into a therapeutic procedure when it was discovered that this technique was capable of completely removing the mucosal layer. The technique is widely used in Japan for the curative treatment of superficial “early” cancers of the gastrointestinal tract. EMR is typically used to remove large flat colon polyps endoscopically without colon surgery. EMR is a leading edge interventional endoscopy procedure available to those motivated to pursue it. The recommendations of the Japanese Society of Digestive Endoscopy include as indications for definitive endoscopic treatment all colonic adenomas and those adenocarcinomas of small size, well differentiated, limited to the mucosa or with invasion of the submucosa lower than 1 μm deep and without lymphatic or vascular invasion. On the other hand, they specify that flat depressed lesions are high metastatic risk ones and recommend to restrict definitive endoscopic resection for lesions smaller than 1 cm diameter [J. Ruiz-Tovar et al., 102. N.° 7, pp. 435-441, 2010]. Basic EMR technique for sessile polyps 1-2 cm in size, or for small flat adenomas smaller than 1 cm, should be within the armamentarium of all colonoscopists. However, effective endoscopic removal of large or complex lesions by EMR can only be achieved by appropriate referral to expert endoscopists skilled in the technique, and all too often patients with lesions that could be removed endoscopically undergo surgery because there is a lack of an appropriate referral pathway. The use of poor endoscopic technique by inexperienced endoscopists may be harmful, as resulting in incomplete removal or major endoscopic complication [How I do it: Removing large or sessile colonic polyps, Brian Saunders MD FRCP, St Mark's Academic Institute].

Unlike techniques that burn or destroy tissue, mucosectomy provides a tissue specimen for surgical pathology. The procedure is curative when two criteria are met:

a) the cancer is superficial, i e , limited to the mucosal layer; and b) the margins of resection are free of tumor.

EMR is performed by first elevating a lesion and its surrounding tissues using a medical solution injected into the submucosa at the site of the lesion, creating a “safety cushion”. The cushion lifts the lesion to facilitate its removal thereby minimizing mechanical or electrocautery damage to the deep layers of the GI tract wall. A snare is placed around the elevated tissue, which is then resected endoscopically by electro coagulation.

Standard EMR methods include:

a. Snare polypectomy;

b. Strip biopsy;

c. EMR with cap technique; and

d. EMR with ligation technique.

Endoscopic submucosal dissection (ESD) has developed in Japan and is being performed in recent years also in large medical centers in USA and Europe. The method employs endoscopic mucosal resection to enable reliable en bloc resection of large and sessile superficial colorectal neoplastic lesions (>10 mm), wherein said technique both reduces residual disease and allows precise pathological evaluation. En bloc resection of neoplastic mucosa is performed by dissecting the sub-mucosal between the mucosa and muscularis propria, after dissection of the per-tumor mucosa. The method involves a high level of technical difficulty, requiring skill and experience, time consuming and carries a relatively high rate of major complication.

ESD procedure is performed by first marking dots on the mucosa around the tumor. A medical solution is then injected into the sub mucosal layer in order to lift the lesion. A mucosal incision is made outside the marking dots and dissection of the sub mucosal layer is performed using special endoscopic electrocautery knives, and en- bloc resection is achieved.

Various treatment instruments for endoscopes have been proposed to assist in ESD and reduce the degree of its technical difficulty.

JP Patent Application No. 2004-275641 discloses a hook knife in which a high-frequency electrode at the tip is formed with a curved rod. By hooking the tip of the hook knife in mucosa tissue and drawing it into a sheath, the mucosa tissue is dissected.

JP Patent Application No. 8-299355 encompasses an IT knife in which an insulator is attached to the tip of an acicular surgical knife so that piercing of muscularis propria is prevented by the insulator.

US Patent application No. US 2009/0247823 is directed to a treatment instrument for an endoscope, which includes a treatment portion having a cutting unit at a tip of an insertion portion that is to be inserted into the body. The main unit of the treatment portion is formed in a saw tooth shape having a peak portion and a valley portion. An electrode plate serving as the cutting unit is provided in the valley portion.

Another currently available tool for ESD procedure is a water-jet HybridKnife for submucosal dissection of mucosal and submucosal lesion in the upper GI tract. The HybridKnife comprises a tip used for setting coagulation markers with safety margins around the targeted lesion. The knife is then positioned close to some of the markers. Activation of the foot-switched controlled water-jet allows submucosal infusion of saline solution for a rapid lifting of the lesion. Circumferential incision of the mucosal layer can be safely performed on top of the submucosal cushion at the periphery of the markers. The HybridKnife is then alternatively used for injection with the water-jet system and cutting as well as for coagulation of visible vessels. The direction of dissection is targeted tangentially to the surface of the lesion at the submucosal layer to minimize the risk of perforation.

US Patent Application No. 2010/0145352 discloses a medical device for removing targeted tissue from a body lumen in a patient. The distal end of the device is placed through a natural orifice in the patient to a location that is proximate to the targeted tissue; deploying a T-anchor fastened to a suture strand through the targeted tissue; deploying a loop anchor into the tissue of the body lumen spaced away from the targeted tissue, whereas the suture strand is slidably received by the loop anchor; applying tension to the suture strand; cutting the tissue at a predetermined depth around the periphery of the targeted tissue; and removing the targeted tissue along with the T-anchor from the body lumen. The tension applied to the targeted tissue maintains the targeted tissue in a raised position and/or allows the physician to manipulate the targeted tissue relative to the tissue that is proximate to it.

International Patent Application No. WO 2006/122279 is directed to apparatus and methods for internal surgical procedures, involving supporting internal body locations, creating submucosal separations (blebs), and/or for resecting mucosal tissue separated from underlying tissue by a bleb.

US Patent Application No. 2007/0260178 discloses an apparatus and methods for performing endoscopic mucosal resection and endoscopic submucosal dissection of tissue, the apparatus comprising catheter having proximal and distal ends and a balloon disposed near the distal end of the catheter. A portion of the distal end of the catheter is configured to be inserted beneath a section of mucosal tissue having a lesion, and the balloon is configured to be inflated to lift the mucosal tissue in an upward direction, thereby facilitating removal of the tissue comprising the lesion.

All of the hereinto mentioned EMR and ESD instruments and techniques require inflation of the sessile lesion. The typical solutions suitable for submucosal injection are shown in FIG. 1. According to the graph presented in FIG. 1, the ability of currently used substances to create the elevation cushion varies significantly among the substances, likewise their stability properties. The ability of the substance to form the cushion depends on the viscosity properties of the substances. The most popular substance, saline (sodium chloride), has low viscosity and as a result a low cushion elevation ability. Saline's cushion is relatively short lived and the mucosal elevation is not as marked as other solutions. Low viscosity of the injected solution may lead to a “balloon deflation effect” that may occur during resection of the lesion. Repeated injections during the resection may be, therefore, required while using saline solution. Another type of substances used for lesion elevation includes highly viscous materials, such as HPMC (hydroxypropyl methylcellulose). As shown in the graph, HPMC provides an elevated cushion for a longer period of time, compared to saline, due to its higher viscosity. However, the major disadvantage of the high viscosity of HPMC is high injection force required to fill the lesion with the elevating material.

US Patent Application No. 2011/0052490 discloses a use of composition comprising purified inverse thermosensitive polymer in an endoscopic procedure for gastrointestinal mucosal resectioning in a mammal. Said invention is further directed to a method of gastrointestinal mucosal resectioning, comprising administering submucosally to a region of a gastrointestinal mucosa in a mammal an effective amount of a composition comprising a purified inverse thermosensitive polymer; and surgically resecting said region of gastrointestinal mucosa. The mucosal elevation obtained with purified inverse thermosensitive polymer is more durable than that obtained with other commonly used substances.

U.S. Pat. No. 7,909,809 is directed to bulking or cushioning agents or material and related medical devices and methods, comprising performing a medical procedure in a tract of a body including injecting a material in a liquid phase proximate a target site between a first tissue layer and a second tissue layer, allowing the material to transition from the liquid phase to the gel phase in response to a raise in temperature of the material to approximately at or above the predetermined temperature, and performing a surgical procedure on the target site. The material may have the liquid phase at temperatures below a predetermined temperature and a gel phase at temperatures approximately at or above the predetermined temperature.

Russian Patent No. 2478344 teaches a method of endoscopic surgery for the treatment of early stomach cancer, including fibrogastroscopic visualization of pathological focus, introduction of endoscopic needle and protrusioning of affected section by injection of solution of liquid, which plays the role of separating film between healthy and pathological tissue of stomach mucosa. As protrusioning liquid used is alcohol solution of Bakelite phenolic resin [—C6H3(OH)—CH2-]n. After that, mucosectomy on the zone of hardened film is performed.

There, however, still exists an unmet need for an improved method and device for inflation of a sessile lesion during EMR and ESD procedures, which would provide controlled long-term shape-sustaining tissue elevation and allow lesion removal with real-time feedback.

The foregoing examples of the related art and limitations related therewith are intended to be illustrative and not exclusive. Other limitations of the related art will become apparent to those of skill in the art upon a reading of the specification and a study of the figures.

### SUMMARY

The present invention is directed to devices and methods for prolonged tissue elevation, which are specifically useful in endoscopic operations. The devices and the methods of the present invention are configured to provide injection of a composition, which is configured to undergo phase transition in response to applied physical or chemical stimuli. The devices and the methods are further configured to controllably induce phase transition of said composition. According to some embodiments of the invention, the composition is injected to a target area, wherein the composition is in a liquid state and is solidified following the controllable application of the stimulus. According to other embodiments, the composition is injected to a target area, wherein the composition is in a solid state, following the controllable application of the stimulus. The liquid composition can be conveniently delivered to the device and/or to the target area, without applying extensive pressure, thereby obviating the use of complicated delivery mechanisms, for example, pumps, while the solid substance contained in the target tissue following the phase transition of the composition allows a substantial and prolonged elevation of the tissue as the solid substance does not diffuse to the contacting tissue and retains the original cushion shape.

Thus, according to one aspect, the present invention provides a method for lifting a first tissue layer with respect to a second tissue layer adjacent thereto, during an endoscopic procedure, the method comprising: delivering a composition configured to undergo phase transition, to a region between the first tissue layer and the second tissue layer; and controllably inducing phase transition of the composition from a liquid state to a solid state thereof.

In some embodiments, the step of delivering the composition comprises delivering the composition in the liquid state thereof. In further embodiments, the step of delivering the composition precedes the step of controllably inducing the phase transition of the composition. In additional embodiments, the method further comprises delivering the composition, in the solid state thereof, to a region between the first tissue layer and the second tissue layer.

In other embodiments, the step of delivering the composition comprises delivering the composition in the solid state thereof. In further embodiments, the step of delivering the composition follows the step of controllably inducing the phase transition of the composition.

According to the preferred embodiments, the step of controllably inducing phase transition of the composition from the liquid state to the solid state comprises controlling an extent of the phase transition of the composition, a proportion of the composition which undergoes phase transition, a position of the composition which undergoes phase transition, a rate of the phase transition of the composition, a duration of the phase transition of the composition or any combination thereof. Each possibility represents a separate embodiment of the invention. The extent of the phase transition of the composition may be defined, inter alia, by the dynamic viscosity and/or hardness of said compositions. Each possibility represents a separate embodiment of the invention. The extent of the phase transition may be further defined by compressibility of the composition. In further embodiments, the extent of the phase transition is defined by leakage or diffusion properties of said composition.

According to certain embodiments, the composition in the solid state thereof does not diffuse from or leak out of the region between the first tissue layer and the second tissue layer. Each possibility represents a separate embodiment of the invention. According to further embodiments, at least a portion of the composition being in the solid state, has a dynamic viscosity above about 60 Pa·s. According to still further embodiments, the composition in the solid state thereof has a dynamic viscosity above about 60 Pa·s. According to some particular embodiments, the dynamic viscosity is above about 80 Pa·s. According to other particular embodiments, the dynamic viscosity is above about 100 Pa·s. According to further particular embodiments, the dynamic viscosity is above about 120 Pa·s. According to additional particular embodiments, the dynamic viscosity is above about 150 Pa·s.

According to further embodiments, at least a portion of the composition is solid following the step of controllably inducing phase transition. According to still further embodiments, the composition is solid following the step of controllably inducing phase transition. According to yet further embodiments, at least a portion of the composition has a dynamic viscosity above about 60 Pa·s, following the step of controllably inducing phase transition. According to still further embodiments, the composition has a dynamic viscosity above about 60 Pa·s, following the step of controllably inducing phase transition. According to other embodiments, the composition in the liquid state thereof has a dynamic viscosity below about 0.15 Pa·s. According to other embodiments, the dynamic viscosity of the composition in the liquid state thereof is below about 0.15 Pa·s at about 37° C.

In some embodiments of the invention, the phase transition of the composition is irreversible.

In some embodiments the step of controllably inducing phase transition of the composition from the liquid state to the solid state is performed repeatedly. In other embodiments, the step of delivering the composition is performed repeatedly. In the preferred embodiments, the step of delivering the composition in the liquid state thereof is performed only once during the endoscopic operation.

According to some embodiments, the step of controllably inducing phase transition of the composition from the liquid state to the solid state comprises providing heating, cooling, electromagnetic radiation, ultrasound radiation or a combination thereof. Each possibility represents a separate embodiment of the invention. In certain embodiments, the step of controllably inducing phase transition of the composition from the liquid state to the solid state comprises providing heating. In further embodiments, the step of controllably inducing phase transition of the composition comprises providing heating to a temperature of about 40° C. to about 85° C. In some embodiments, the heating is to a temperature of above about 40° C. In further embodiments, the heating is to the temperature of above about 50° C. In additional embodiments, the heating is to the temperature of above about 60° C.

According to some embodiments, the step of controllably inducing phase transition of the composition from the liquid state to the solid state comprises inducing phase transition of the composition to obtain a defined solid structure. In some embodiments, said defined solid structure comprises a solid skeleton. In further embodiments, the defined solid structure further comprises liquid and/or gel composition. In some embodiments, the delivered composition has a bulk portion and a periphery portion, wherein said periphery portion contacts the first tissue and/or the second tissue. Said composition may be in the liquid state or a solid state. Each possibility represents a separate embodiment of the invention. In some embodiments, the step of controllably inducing phase transition of the composition to the solid state comprises inducing phase transition of the bulk portion of the composition to a higher extent as compared to the periphery portion of the composition. In other embodiments, the bulk portion of the composition has a higher dynamic density than the periphery portion of the composition.

According to some embodiments, the controllable inducing of the phase transition of the composition provides prolonged elevation of the first tissue layer with respect to the second tissue layer. According to particular embodiments, the composition in the solid state thereof, disposed between the first tissue layer and the second tissue layer provides prolonged elevation of the first tissue layer with respect to the second tissue layer. In some embodiments, said prolonged elevation is maintained for above about one hour. In other embodiments, said prolonged elevation is maintained for above about two hours. In further embodiments, said prolonged elevation is maintained for above about three hours. In yet further embodiments, said prolonged elevation is maintained for above about four hours. In yet further embodiments, said prolonged elevation is maintained for above about five hours.

In further embodiments, the controllable inducing of the phase transition of the composition provides prolonged elevation of the first tissue layer with respect to the second tissue layer of from about 3 mm to about 18 mm According to particular embodiments, the composition in the solid state thereof, disposed between the first tissue layer and the second tissue layer provides prolonged elevation of the first tissue layer with respect to the second tissue layer of from about 3 mm to about 18 mm In some embodiments, the elevation is of at least about 5 mm In further embodiments, the elevation is of at least about 8 mm.

In additional embodiments, the controllable inducing of the phase transition of the composition provides patching of the region between the first tissue and the second tissue following the endoscopic procedure. According to particular embodiments, the composition in the solid state thereof, disposed between the first tissue layer and the second tissue layer provides patching of the region between the first tissue and the second tissue following the endoscopic procedure.

According to further embodiments, the composition configured to undergo phase transition comprises a thermo-sensitive material, selected from the group consisting of proteins, hydrocolloids and combinations thereof. Each possibility represents a separate embodiment of the invention. The protein may be selected from the group consisting of bovine serum albumin, β-lactoglobulin, egg albumin, ovalbumin, human serum albumin, collagen and combinations thereof. Each possibility represents a separate embodiment of the invention. The hydrocolloid may be selected from the group consisting of guar gum, gum arabic, agar-agar, locust bean gum, brown algae, pectin, pectinate, carrageenan, xanthan, alginate, alginic acid, polygalacturonate, glacturonic acid, galacturonate, mannuronic acid, mannurate, gellan gum, starch, modified starch, cellulose, carboxymethyl cellulose, arabinoxylan, curdlan, gelatin, β-glucan and combinations thereof. Each possibility represents a separate embodiment of the invention.

The composition may further comprise an additive. In some embodiments, the additive comprises a stabilizer, a color indicator, adhesion controller or a combination thereof. Each possibility represents a separate embodiment of the invention. In some embodiments, the method comprises the step of tracking color change of the composition upon the phase transition thereof, which is indicative of the extent of the phase transition, the proportion of the composition which underwent phase transition or a combination thereof. Each possibility represents a separate embodiment of the invention.

The stabilizer may be selected from the group consisting of polyoxazoline, poloxamers, polyvinylpyrrolidone (PVP) and combinations thereof. Each possibility represents a separate embodiment of the invention. The color indicator may be selected from the group consisting of pH indicators, carotenoids and combinations thereof. Each possibility represents a separate embodiment of the invention. The adhesion controller may be selected from the group consisting of phospholipids, monoglycerides and combinations thereof. Each possibility represents a separate embodiment of the invention. The composition may further include a suitable solvent, excipient or a combination thereof. Each possibility represents a separate embodiment of the invention.

In some embodiments, the methods of the present invention are for use in an endoscopic surgery selected from endomucosal resection (EMR) or endoscopic submucosal dissection (ESD).

In another aspect, there is provided a device for lifting a first tissue layer with respect to a second tissue layer adjacent thereto, during an endoscopic procedure, the device comprising: an injection module having an elongate body, a proximal region and a distal region, wherein the distal region comprises at least one outlet, configured to deliver a composition configured to undergo phase transition, to a region between the first tissue layer and the second tissue layer; and a phase transition module, configured to controllably induce phase transition of the composition from a liquid state to a solid state thereof. In an additional aspect, there is provided a kit for lifting a first tissue layer with respect to a second tissue layer adjacent thereto, during an endoscopic procedure, the kit comprising a composition configured to undergo phase transition from a liquid state to a solid state; a phase transition module, configured to controllably induce phase transition of the composition from a liquid state to a solid state and, optionally, an injection module having an elongate body, a proximal region and a distal region, wherein the distal region comprises at least one outlet, configured to deliver the composition to a region between the first tissue layer and the second tissue layer. In the preferred embodiments, the phase transition module is enclosed within the injection module. The phase transition module is preferably disposed in the distal region of the injector module.

In some embodiments, the injection module is configured to deliver the composition, in the liquid state thereof, and the phase transition module is configured to controllably induce phase transition of the delivered composition from the liquid state to the solid state thereof.

In other embodiments, the phase transition module is configured to controllably induce phase transition of the composition, disposed within the device, from the liquid state to the solid state thereof and the injection module is configured to deliver the composition, in the solid state thereof.

In additional embodiments, the injection module is configured to deliver the composition, in the liquid state thereof and/or in the solid state thereof, and the phase transition module is configured to controllably induce phase transition of the delivered composition or of the composition disposed within the device, from the liquid state to the solid state.

In further embodiments, the device and/or a transition module is configured to allow controlling an extent of the phase transition of the composition, a proportion of the composition which undergoes phase transition, a position of the composition which undergoes phase transition, a rate of the phase transition of the composition, a duration of the phase transition of the composition or any combination thereof. Each possibility represents a separate embodiment of the invention. According to the preferred embodiments, the device and/or a transition module is configured to allow controlling a position of the composition which undergoes phase transition within the region between the first tissue layer and the second tissue layer.

In some embodiments, the phase transition module is configured to provide heating, cooling, electromagnetic radiation or ultrasound radiation of the delivered composition. Each possibility represents a separate embodiment of the invention. In particular embodiments, the phase transition module provides heating. The heating may be to a temperature of above about 40° C., such as above about 60° C. or above about 60° C. Each possibility represents a separate embodiment of the invention.

In some embodiments, the phase transition module comprises at least one electrode, connected to a power source. In some particular embodiments, the phase transition module comprises bipolar electrodes, exposed at opposite sides of the distal region of the phase transition module, wherein the exposed electrodes are configured to face the delivered composition. In other particular embodiments, the phase transition module comprises concentric or planar electrodes. The device may further comprise a cutting means disposed in the distal region of the phase transition module and/or of the injection module.

In further embodiments, the distal region of the injection module further comprises an orientation indicator, configured to indicate the spatial orientation of the injection module distal region. In yet further embodiments, the distal region of the injection module further comprises a phase transition indicator, configured to provide indication of the phase transition state of the composition.

According to further embodiments, the elongate body of the injection module comprises a sliding surface, configured to facilitate smooth sliding of the injection module upon the second tissue layer. The injection module may further comprise a plurality of outlets, configured to stabilize the injection module spatial orientation during the composition delivery.

In some embodiments, the device of the present invention comprises a tube in a fluid-flow connection with the injection module.

In further embodiments, the device further comprises an actuator, configured to assist the delivery of the composition, in the solid state thereof, to the region between the first tissue layer and the second tissue layer. The device may further comprise a dosing module, configured to be in a fluid-flow connection with the proximal region of the device and further configured to provide a metered delivery of the composition to the region between the first tissue layer and the second tissue layer. In some embodiments, said composition is in the solid state thereof.

In the preferred embodiments, the device is configured to be operated through an endoscope. In some embodiments, the device is for use in the endoscopic procedure selected from endomucosal resection (EMR) or endoscopic submucosal dissection (ESD). Thus, according to some embodiments, there is provided a use of said device in the endoscopic procedure, comprising: inserting the device between the first tissue layer and the second tissue layer; delivering, using the injection module, the composition configured to undergo phase transition, to the region between the first tissue layer and the second tissue layer; and controllably inducing, using the injection module, the phase transition of the composition configured to undergo phase transition from the liquid state to the solid state thereof.

According to further embodiments, there is provided a method of lifting a first tissue layer with respect to a second tissue layer adjacent thereto, during an endoscopic procedure, the method comprising: inserting the device of the present invention to the region between the first tissue layer and the second tissue layer; delivering, using the injection module, the composition configured to undergo phase transition, to the region between the first tissue layer and the second tissue layer; and controllably inducing, using the injection module, the phase transition of the composition configured to undergo phase transition from the liquid state to the solid state thereof.

According to additional embodiments, there is provided an endoscopic system, comprising an endoscope and a device for lifting a first tissue layer with respect to a second tissue layer, during an endoscopic procedure, wherein the device is operable through the endoscope. According to further embodiments, the endoscopic system comprises a control unit. The control unit may comprise a user interface, configured to provide real time information and/or control over the device position and spatial orientation. The user interface may further be configured to provide real time information and/or control over the extent of the phase transition of the composition, the proportion of the composition which undergoes phase transition, the position of the composition which undergoes phase transition, the rate of the phase transition of the composition, the duration of the phase transition of the composition or any combination thereof. Each possibility represents a separate embodiment of the invention.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. It is intended that the embodiments and figures disclosed herein are to be considered illustrative rather than restrictive. The figures are listed below.

FIG. 1 represents an effect of different injectable solutions on tissue elevation height and time;

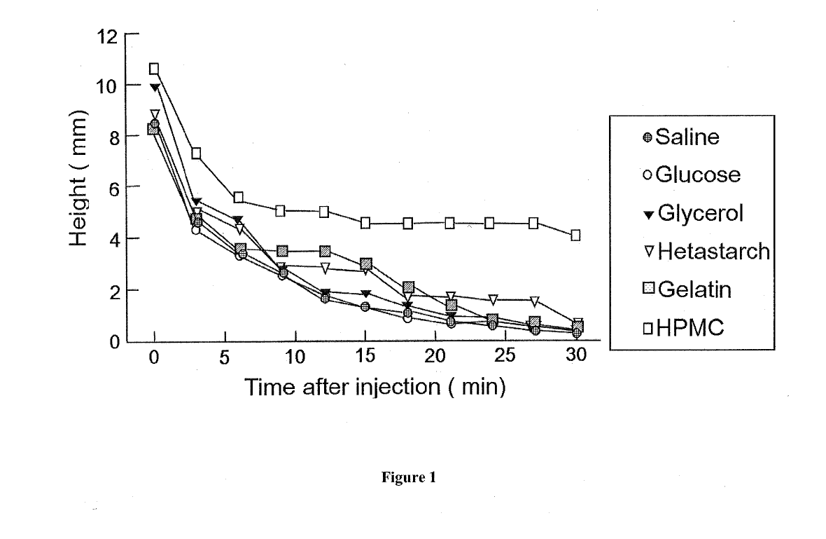

FIG. 2 schematically illustrates an endoscopic system;

FIG. 3A schematically illustrates an endoscope;

FIG. 3B schematically illustrates a detailed view of a control box of an endoscope;

FIG. 4A schematically illustrates an endoscope;

FIG. 4B schematically illustrates a detailed view of an integral composition container;

FIG. 5 schematically illustrates an endoscope system display output;

FIG. 6A schematically illustrates an intruder, connected to a tube;

FIG. 6B schematically illustrates the intruder, connected to the tube, wherein the intruder is shown in its upturned position;

FIG. 6C schematically illustrates the intruder connected to the tube, wherein the intruder includes inspection LED;

FIG. 7A schematically illustrates the intruder, connected to the tube, wherein the intruder includes injection outlets;

FIG. 7B schematically illustrates the intruder, connected to the tube, wherein the intruder includes injection outlets and wherein the intruder is shown in its upturned position;

FIG. 8A schematically illustrates a heating module;

FIG. 8B schematically illustrates a cross-section view of the heating module;

FIG. 9A schematically illustrates an integrated intruder, combining the intruder (presented in FIGS. 7A and 7B) and the heating module (presented in FIG. 8A and 8B);

FIG. 9B schematically illustrates the integrated intruder in its upturned position.

FIG. 9C schematically illustrates the integrated intruder internal view;

FIG. 9D schematically illustrates a cross-section view of the integrated intruder;

FIG. 10A schematically illustrates a cross-section view of an integrated retractable intruder;

FIG. 10B schematically illustrates the integrated retractable intruder in its upturned position;

FIG. 10C schematically illustrates a cross-section view of the integrated retractable intruder;

FIG. 11 schematically illustrates an operation mode of the integrated intruder wherein the composition configured to undergo phase transition is delivered to the target site in the liquid state thereof;

FIGS. 12A-12B schematically illustrate a tissue elevation process: FIG. 12A depicts an injectable composition injection and FIG. 12B depicts phase transition of the injected composition.

FIG. 13 schematically illustrates a cross-section view of an intruder, including concentric electrodes;

FIG. 14A schematically illustrates an intruder, including parallel electrodes;

FIG. 14B schematically illustrates a cross-sectional view of the intruder, including parallel electrodes;

FIG. 15A schematically illustrates a dosing module and storage chamber;

FIG. 15B schematically illustrates a detailed view of the dosing module;

FIG. 16A schematically illustrates an actuator assisted intruder, wherein a piston valve is in retracted position;

FIG. 16B schematically illustrates the actuator assisted intruder, wherein the piston valve is in extended position; and

FIG. 17 schematically illustrates an operation mode of the intruder, wherein the composition configured to undergo phase transition is delivered to the target site in the solid state thereof.

### DETAILED DESCRIPTION

The present invention is directed to a device, configured to provide a sustainable elevation of the target tissue. The device may be operated through an endoscope, be connected to an endoscope or may be an integral part of an endoscope. Each possibility represents a separate embodiment of the invention. The present invention is further directed to a method of performing an endoscopic operation, comprising providing a sustained elevation of the target tissue. Said endoscopic procedure may be carried out in a similar way to any known endoscopic procedure. After identifying the targeted area, the physician injects (singularly or repeatedly) an injectable composition, wherein the composition is configured to undergo phase transition as a result of any physical or chemical action such as, but not limited to, heating, cooling, electromagnetic radiation, ultrasound, or chemical reaction. According to some embodiments, the phase transition is a transition from a liquid state to a solid state. The method of the present invention is configured to allow to controllably facilitate the phase transition of said injectable composition. In further embodiments, the device of the present invention is configured to controllably facilitate the phase transition of said injectable composition. According to some particular embodiments, the method provides injection of said injectable composition, in the liquid phase thereof, into the target area. According to other particular embodiments, the injectable composition is delivered to the target area in the solid state thereof. According to some embodiments, the injectable composition is configured to undergo phase transition prior to contacting the target area. The phase transition of the composition may be performed according to some embodiments of the invention, following the delivery thereof to the target area. According to other embodiments, the method of the present invention includes phase transition of said composition prior to the delivery thereof to the target area.

In some embodiments, the composition, injected by the device, while still in the liquid form, separates layers of tissues inside the target area. Upon exposure of the injectable composition to the selected physical or chemical action, facilitated by the device, the injectable composition solidifies, forming a disjunctive layer between two or more layers of the target tissue in the target area. The disjunctive layer produces a sufficiently wide gap, allowing to safely remove one or more layers of the target tissue using any known removal method. Without wishing to being bound by any specific theory or mechanism of action, the injectable composition, when delivered in a liquid state thereof, occupies the desired volume between the first and the second tissues, allowing substantially full separation between the first and the second tissues. In some embodiments, the liquid composition is distributed uniformly between the first and the second tissues, along the target area. Following solidification, the composition retains its shape, preserving the substantially full separation of the tissues. In some embodiments, the term “substantially full separation” relates to the separation along the region between the tissue layers, occupied by the liquid composition.

According to other embodiments, upon exposure of the composition to the selected physical or chemical action facilitated by the lifting device, the composition solidifies, and is ejected in the solid state thereof, by the lifting device into the target area. The composition, delivered by the device, following the phase transition thereof, separates layers of tissues inside the target area. According to some embodiments, the solidified composition forms a disjunctive layer between two or more layers of the target tissue in the target area. The disjunctive layer produces a sufficiently wide gap, allowing to safely remove one or more layers of the target tissue using any known removal method.

Thus, according to one aspect, there is provided a method for lifting a first tissue layer with respect to a second tissue layer adjacent thereto, during an endoscopic procedure, the method comprising: delivering a composition configured to undergo phase transition, to a region between the first tissue layer and the second tissue layer; and controllably inducing phase transition of the composition, configured to undergo phase transition, from the liquid state to the solid state thereof. In some embodiments, the method includes (a) delivering the composition configured to undergo phase transition, in the liquid state thereof, to the region between the first tissue layer and the second tissue layer, thereby lifting the first tissue layer with respect to the second tissue layer; and (b) controllably inducing phase transition of the delivered composition to the solid state, thereby further lifting and stabilizing the elevation of the first tissue layer with respect to the second tissue layer, wherein step (b) is performed following step (a). In other embodiments, the method includes (i) controllably inducing phase transition of the composition configured to undergo phase transition, from the liquid state to the solid state thereof; and (ii) delivering the composition configured to undergo phase transition, in the solid state thereof, to the region between the first tissue layer and the second tissue layer, thereby lifting the first tissue layer with respect to the second tissue layer, wherein step (ii) is performed following step (i).

As used herein the terms “elevation” and/or “lifting”, which may be used interchangeably, refer to displacing a first tissue layer with respect to a second tissue layer, adjacent thereto, by filling a void between the layers with a physical barrier.

As used herein, the term “first tissue layer” refers to a tissue layer targeted for removal. The non-limiting examples of the “first tissue layer” include a malignant tissue or a sessile lesion.

As used herein, the term “second tissue layer” refers to a tissue layer, contacting the tissue layer targeted for removal. The “second tissue layer” may refer, for example, to a healthy tissue.

In particular embodiments, the first tissue layer and the second tissue layer refer to the tissue of the gastrointestinal tract.

The term “solid state” as used in some embodiments of the invention, refers to a physical state of the composition in which at least a portion of the composition is solid. According to further embodiments, “solid state” refers to a physical state, wherein at least a portion of the composition has a dynamic viscosity characteristic of a solid composition. The solid composition typically has a dynamic viscosity in the range of about 60 to about 250 Pa·s, such as, for example, about 60 to about 100 Pa·s, about 100 to about 150 Pa·s, 150 to about 200 Pa·s or about 200 to about 250 Pa·s. Each possibility represents a separate embodiment of the invention. Said portion of the composition may comprise a 10%, 20%, 30%, 40%, 50%, 60%, 70%, 80%, 90% or 100% of the composition. Each possibility represents a separate embodiment of the invention. According to yet further embodiments, “solid state” refers to a physical state of the composition, having a net dynamic viscosity and/or hardness, sufficient to provide a prolonged elevation of the first tissue layer with respect to the second tissue layer for above about one hour. According to still further embodiments, “solid state” refers to a physical state of the composition, having a net dynamic viscosity and/or hardness which enables a prolonged elevation of the first tissue layer with respect to the second tissue layer for above about one hour. According to yet further embodiments, “solid state” refers to a physical state of the composition, having a net dynamic viscosity and/or hardness which enables lifting of the first tissue layer with respect to the second tissue layer of from about 3 mm to about 18 mm, such as, for example, from about 5 mm to about 15 mm or from about 8 mm to about 12 mm Each possibility represents a separate embodiment of the invention. According to still further embodiments, “solid state” refers to a physical state of the composition, having a net dynamic viscosity and/or hardness which prevents diffusion and/or leakage of the composition out of the region between the first and the second tissue layer.

The term “liquid state” as used in some embodiments of the invention, refers to a physical state of the composition in which the composition is liquid. According to some embodiments, “liquid state” refers to a physical state, wherein the composition has a dynamic viscosity characteristic of a liquid composition. The liquid composition typically has a dynamic viscosity in the range of about 0.01 to about 0.15 Pa·s.

In some embodiments, the transition from the liquid phase to the solid phase proceeds through the formation of a gel. In further embodiments, the dynamic viscosity of said gel is below about 50 Pa·s.

In some embodiments, the phase transition of the composition from the liquid state to the solid state in irreversible. In other embodiments, the phase transition is reversible. The phase transition of the composition from the liquid state to the solid state may include solidification, hardening, denaturation of the composition or any combination thereof. Each possibility represents a separate embodiment of the invention. In the preferred embodiments, the phase transition includes increase in the dynamic viscosity of the composition.

According to further embodiments, at least a portion of the composition following phase transition has a dynamic viscosity in the range of about 60 to about 250 Pa·s. In these embodiments, at least a portion of the composition following phase transition is solid. In some embodiments, the composition following the phase transition includes a combination of the solid composition and a liquid composition. In some embodiments, the composition following the phase transition includes a combination of the solid composition, a gel composition and a liquid composition. In other embodiments, the composition following phase transition is substantially completely solid. In other embodiments, the composition following phase transition is devoid of the liquid and/or gel composition.

According to further embodiments, the dynamic viscosity of the composition configured to undergo phase transition, in the solid state thereof, is in the range of about 60 to about 250 Pa·s. In some embodiments, the dynamic viscosity of the composition in the solid state thereof, is in the range of about 60 to about 100 Pa·s, of about 100 to about 150 Pa·s, of 150 to about 200 Pa·s or of about 200 to about 250 Pa·s. According to additional embodiments, the dynamic viscosity of the composition configured to undergo phase transition, in the liquid state thereof, is in the range of about 0.01 to about 0.15 Pa·s.

Without wishing to being bound by any specific theory or mechanism of action, the solid substance contained in the target tissue following the phase transition of the composition allows a substantial and prolonged elevation of the tissue, as the solid substance does not diffuse to the contacting tissue or leak out of the region between the first and the second tissue layer. Thus, a composition in the solid state thereof, wherein at least a portion of the composition is solid, is configured to provide a substantial and prolonged elevation of the tissue. In some embodiments, the composition following phase transition retains the original shape thereof for at least about one hour. In some embodiments, the original shape of the composition in the solid state retains for about 2, 3, 6, 9 or 12 hours. According to some embodiments, the device is configured to provide lifting of the first tissue layer with respect to the second tissue layer of from about 3 mm to about 18 mm, preferably from about 5 mm to about 15 mm, even more preferably from about 8 mm to about 12 mm The method and/or device of present invention provide a physician with a safe and efficient tissue lifting procedure. Said lifting procedure may be used in any endoscopic tissue removal technique, such as, for example, in EMR-en-block, EMR-piecemeal, ESD or a combination thereof. Each possibility represents a separate embodiment of the invention. In some embodiments the removal technique can be varied throughout the operation or can be performed in several acts.

According to some preferred embodiments, the method and/or the device of the present invention provide post-operation patching of the tissue, which remains after the dissection procedure. Thus, in some embodiments, the method and/or the device of the present invention provide patching of the second tissue layer. Said patching is configured to create an additional temporary layer and reinforce and protect the tissue. The patching allows increasing the physician safety and preventing post-operation complications, such as, for example, perforation or inflammation. The patching effect may last for as long as about 1 day, 2 days, 3 days, 4 days or 5 days. Each possibility represents a separate embodiment of the invention. Following this time, the patch is destroyed by the enzymatic activity of the organism and removed from the subject's body.

In the preferred embodiments of the invention, the method and/or the device provide controllable phase transition of the composition configured to undergo phase transition. Controllable inducing of the phase transition may include controllable exposure of the composition to the means, facilitating the phase transition, such as, but not limited to heating, cooling, electromagnetic radiation, ultrasound radiation or a combination thereof. In some embodiments, the phase transition is controlled by the type of the phase transition facilitation means. In other embodiments, the phase transition is controlled by time of the exposure to the phase transition facilitation means. In further embodiments, the phase transition is controlled by the distance between the phase transition facilitating means and the composition. In other embodiments, the phase transition is controlled by other phase transition facilitation parameters, such as, but not limited to temperature, DC voltage, DC current, AC voltage, AC current, AC current type, AC current frequency, AC current amplitude, AC current waveform, electromagnetic radiation frequency, electromagnetic radiation amplitude, electromagnetic radiation waveform, ultrasound radiation frequency, ultrasound radiation amplitude, ultrasound radiation waveform of the phase transition facilitating means or a combination thereof. Each possibility represents a separate embodiment of the invention

In some embodiments, the controllable inducing of the phase transition of the composition includes heating. In particular embodiments, the composition is heated to a temperature of about 40° C. to about 85° C., more specifically, from about 50° C. to about 75° C. In some embodiments, the controllable inducing of the phase transition of the composition includes application of electrical power in the range from about 7 Watt to about 140 Watt, more specifically, from about 20 Watt to about 100 Watt, more specifically, from about 50 Watt to about 70 Watt.

In some embodiments, controllable inducing of the phase transition includes controlling an extent of the phase transition of the composition. Without wishing to being bound by any specific theory or mechanism of action, the method of the present invention allows control over the dynamic viscosity and/or hardness of the composition configured to undergo phase transition. The term “extent of the phase transition”, as used herein, refers to the deviation of the dynamic viscosity of the composition from the dynamic viscosity of the composition in the liquid phase, wherein the substantially full phase transition can be defined as the change in the dynamic viscosity of the 100% of the composition from the dynamic viscosity of the liquid composition to the dynamic viscosity of the solid composition. Hence, the extent of the phase transition may be controlled to provide a 10%, 20%, 30%, 40%, 50%, 60%, 70%, 80%, 90% or 100% of substantially full phase transition of the composition. In some embodiments, the substantially full phase transition is defined as the change from the dynamic viscosity of the liquid composition to the minimal dynamic viscosity of the solid composition. In other embodiments, the substantially full phase transition is defined as the change from the dynamic viscosity of the liquid composition to the maximal dynamic viscosity of the solid composition.

In further embodiments, controllable inducing of the phase transition includes controlling a proportion of the composition which undergoes phase transition. For example, 10%, 20%, 30%, 40%, 50%, 60%, 70%, 80%, 90% or 100% of the composition may undergo phase transition. In some embodiments, the method and/or device of the present invention provide control over the extent of the phase transition of the composition, the proportion of the composition, which undergoes phase transition or a combination thereof.

In further embodiments, controllable inducing of the phase transition includes controlling a position of the composition which undergoes phase transition. In some embodiments, the composition is located within the region between the first tissue layer and the second tissue layer. In other embodiments, the composition is located within the device of the present invention. According to particular embodiments of the present invention, the controllable inducing of the phase transition includes controlling a position of the composition relative to the first and/or to the second tissue layers. According to further embodiments, the composition can be partitioned into a plurality of regions, wherein phase transition of each region can be induced separately.

In some embodiments, the phase transition of the composition is uniform along the target area. In other embodiments, the phase transition is non-uniform, providing varying dynamic viscosity and/or hardness along the target area. Without wishing to being bound by any specific theory or mechanism of action, the non-uniform phase transition of the composition may be used to tailor the composition parameters, such as, but not limited to adhesion or patching effect, to the particular needs. For example, the periphery of the composition, contacting the first and/or the second tissue, may have a lower dynamic viscosity following phase transition, than the bulk of the composition. Without wishing to being bound by any specific theory or mechanism of action, the lower viscosity of the composition, which underwent phase transition, provides improved adhesion of the composition to the first and/or the second tissue, thus providing a patching effect.

In further embodiments, the method/and or the device of the present invention allow controllably inducing phase transition of the composition, to obtain a defined solid structure. For example, only a portion of the composition can be exposed to the phase transition facilitating means, to provide a solid skeleton having a desired shape, wherein the rest of the composition remains in the liquid state thereof, such that the solid skeleton is surrounded by the liquid composition. In other embodiments, the skeleton is surrounded by the composition having a different dynamic viscosity than the solid skeleton. In other embodiments, the skeleton is surrounded by the composition having a lower dynamic viscosity than the solid skeleton.

In some embodiments, the method and/or device of the present invention provide control over the extent of the phase transition of the composition, the proportion of the composition, which undergoes phase transition, the position of the composition which undergoes phase transition or a combination thereof.

In further embodiments, the step of controllably inducing the phase transition of the composition includes controlling the rate of the phase transition, the duration of the phase transition or a combination thereof. According to some embodiments, the duration of the phase transition is from about 1 sec to about 1 minute, more specifically, from about 1 sec to about 30 sec, more specifically, from about 5 sec to about 20 sec. In some embodiments, controllable inducing of the phase transition of the composition includes controlling the number of repetitions of said phase transition induction.

In some embodiments, the method and/or the device provide controllable delivery of the composition. The step of controllably delivering the composition configured to undergo phase transition to the region between the first tissue layer and the second tissue layer can include control over the position of the delivered composition, rate of the delivery, time of the delivery, the amount of the composition, number of times the composition is delivered or a combination thereof. In the preferred embodiments, the composition in the liquid state thereof is delivered to a target region only once during the endoscopic procedure. As the method and/or device of the present invention provide sustained elevation of the first tissue layer with respect to the second tissue layer, repeated injections of the liquid composition are generally not required, when using the method and/or device of the present invention. In some embodiments, the injection of the composition in the solid state thereof is performed multiple times.

According to some embodiments, the composition configured to undergo phase transition includes an active ingredient, such as, but not limited to a thermo-sensitive material or a chemically-active material. According to further embodiments, the thermo-sensitive material is configured to transform from a liquid state to a solid phase as a result of heating, cooling or a combination thereof. According to further embodiments, the chemically-active material is configured to transform from a fluid state to a solid state as a result of a chemical reaction. According to further embodiments, the thermo-sensitive material is selected from the group consisting of a protein, a hydrocolloid and a combination thereof. Each possibility represents a separate embodiment of the invention. According to particular embodiments, the proteins and/or hydrocolloids are characterized by the ability thereof to transform from a liquid state to a solid state as a result of heating.

According to some embodiments, the composition is hardenable (e.g. cured, cross-linked or set) by physical or chemical means. Chemical means include contact with a hardening, e.g. cross-linking agent or a gel-forming agent. Physical means may comprise heating and/or radiation. In some embodiments, the composition is configured to be hardened after it has been injected into the target area. In other embodiments, the composition is configured to be hardened before it has been injected into the target area.

The protein may be milk derived (a lactoprotein), such as casein or whey; vegetable derived, such as soy; cereal derived, for example from maize, corn or wheat, such as gluten; or egg derived (e.g. ovoprotein). Each possibility represents a separate embodiment of the invention. Collagen can also be used in the compositions configured to undergo phase transition.

The protein may thus be a heat-sensitive protein such as one that denatures (or solidifies, or becomes water-insoluble) when heated, for example egg derived albumin or whey derived β-lactoglobulin. The protein may alternatively or additionally be cross-linkable, and can therefore assist in the hardening.

According to further embodiments, the hydrocolloid is hardenable. According to yet further embodiments, the hydrocolloid is hardenable by either physical or chemical means. According to still further embodiments, the hydrocolloid is cross-linkable and/or gellable. Hardening may be by cross-linking that is irreversible (e.g. physical or covalent linking) or by a reversible technique (e.g. ionic linking).

The hydrocolloid is preferably a polysaccharide. Suitable hydrocolloids include guar-gum, gum arabic, agar-agar, locust bean gum, brown algae, pectin, pectinate, carrageenan, xanthan, alginate, alginic acid, polygalacturonate, glacturonic acid, galacturonate, mannuronic acid, mannurate, gellan gum, starch, modified starch, cellulose, carboxymethyl cellulose, arabinoxylan, curdlan, gelatin, β-glucan or combinations thereof. Each possibility represents a separate embodiment of the invention.

According to a certain embodiment, the protein is selected from the group consisting of albumin and β-lactoglobulin. Each possibility represents a separate embodiment of the invention.

According to additional embodiments, the hydrocolloid is selected from the group consisting guar-gum, agar-agar, locust bean gum, brown algae, and pectin. Each possibility represents a separate embodiment of the invention.

According to further embodiments, the composition further includes an additive, wherein the additive may include a stabilizer, a color indicator or a phase-transition trigger.

The stabilizer can stabilize the fluid phase of the active ingredient prior to phase transition and/or to induce the phase transition of the active ingredient. According to some embodiments, stabilizers may include polyoxazoline, poloxamers, gelatin, oil, polyvinylpyrrolidone (PVP), or a combination thereof.

According to some embodiments, the additives further include a color indicator, configured to allow control over the phase transition. The color indicators correlate with the phase-transition level and therefore with the target tissue elevation degree.

The color indicators may include pH indicators, carotenoids or a combination thereof. The pH indicators suitable for use in the injectable composition include pH indicators with the transition pH range from about 7.9 to about 6.8. The suitable pH indicators include anthocyanin, phenolphthalein or azo-dyes. The pH indicator may be present in the injectable composition in an amount from about 100 to about 10,000 ppm. According to some embodiments, anthocyanin is present in the injectable composition in the concentrations ranges of 100-500 ppm. According to other embodiments, phenolphthalein is present in the injectable composition in concentrations range of 1,000-10,000 ppm. According to additional embodiments, azo-dye is present in the composition in concentrations range of 200-1,000 ppm.

The carotenoids suitable for use in the composition include carotenoids which change a color or a hint thereof upon thermal application. According to some embodiments, the carotenoids usable in the composition include pigments extracted from vegetables, such as but not limited to, tomato and carrot. According to additional embodiments, suitable carotenoids include lycopene or β-carotene.

The carotenoid may be present in the composition in an amount from about 2 to about 10 ppm. According to some embodiments, lycopene is present in the composition in concentrations range of 2-10 ppm. According to other embodiments, β-carotene is present in the composition in concentrations range of 2-10 ppm. Those dyes are temperature sensitive and the changes there tone/color from red and or orange tone into colorless at after phase transition process is complete.

The method of the present invention may thus further include a step of tracking color change of the composition upon the phase transition thereof, which is indicative of the extent of the phase transition, the proportion of the composition which underwent phase transition or a combination thereof.

In further embodiments, the composition includes an adhesion controller, such as, but not limited to phospholipids or monoglycerides. Without wishing to being bound by any specific theory or mechanism of action, the adhesion controller is configured to induce patching effect and/or to prevent adhesion of the composition to the device of the present invention.

According to further embodiments, the composition may include a phase- transition trigger such as a cross-linking agent or a gel-forming agent. According to still further embodiments, the composition may further include a solvent, such as, but not limited to, water or saline. The compositions may further include one or more excipients, carriers or buffers. One non-limiting example of a buffer is a phosphate buffer.

The composition configured to undergo phase transition, including an active agent and, optionally, an additive, allows a long-term elevation of the target tissue.

According to some embodiments, the injectable composition has a phase transition temperature in the range from about 40° C. to about 85° C., more specifically, from about 50° C. to about 75° C. According to further embodiments, the composition is susceptible to phase transition from fluid phase to solid phase at the applied electrical power in the range from about 7 Watt to about 140 Watt, more specifically, from about 20 Watt to about 100 Watt, more specifically, from about 50 Watt to about 70 Watt. According to additional embodiments, the composition undergoes phase transition at a time period from about 1 sec to about 1 minute, more specifically, from about 1 sec to about 30 sec, more specifically, from about 5 sec to about 20 sec. According to some embodiments, the composition has an electric resistivity in the range from about 20 to about 500 A²·s⁴·kg⁻¹·m⁻³. According to further embodiments, the composition is characterized by a color or/and a transparency change, induced by the phase transition.

According to some embodiments the thermo-sensitive material is present in the composition in an amount from about 0.1% (w/w) to about 30% (w/w). According to some embodiments, the protein is present in the composition in an amount from about 1% (w/w) to about 30% (w/w). According to further embodiments, the protein is present in the composition in an amount from about 5% (w/w) to about 25% (w/w). According to still further embodiments, the hydrocolloid is present in the composition in an amount from about 0.1% (w/w) to about 30% (w/w). According to yet further embodiments, the hydrocolloid is present in the composition in an amount from about 1% (w/w) to about 5% (w/w). According to still further embodiments, the stabilizer is present in the composition in an amount from about 1% (w/w) to about 8% (w/w). According to yet further embodiments, the adhesion controller is present in the composition in an amount of up to about 3% (w/w).

According to some embodiments, the composition includes a protein and a stabilizer. According to other embodiments, the composition includes a hydrocolloid and a stabilizer. According to other embodiments, the composition includes a protein, a hydrocolloid and a stabilizer.

According to some embodiments, the composition includes albumin, gelatin and saline. According to other embodiments, the composition includes pectin and saline. According to further embodiments, the composition includes pectin, albumin and saline. According to additional embodiments, the composition includes guar gum and saline. According to further embodiments, the composition includes guar gum, albumin and saline. According to yet further embodiments, the composition includes albumin, PVP and saline. According to still further embodiments, the composition includes modified starch, gelatin and saline.

According to some embodiments, the composition includes albumin, gelatin and phosphate buffer. According to other embodiments, the composition includes pectin and phosphate buffer. According to further embodiments, the composition includes pectin, albumin and phosphate buffer. According to additional embodiments, the composition includes guar gum and phosphate buffer. According to further embodiments, the composition includes guar gum, albumin and phosphate buffer.

According to yet further embodiments, the composition includes albumin, PVP and phosphate buffer. According to still further embodiments, the composition includes modified starch, gelatin and phosphate buffer.

According to some embodiments, the composition includes 1-2% (w/w) gelatin, 12-28% (w/w) albumin and saline. According to other embodiments, the composition includes 0.5-2% (w/w) pectin and saline. According to some embodiments, the composition includes 0.1-1% (w/w) pectin, 12-28% (w/w) albumin and saline.

According to some embodiments, the composition includes 0.5-2% (w/w) guar gum and saline. According to other embodiments, the composition includes 0.3-1% (w/w) pectin, 12-28% (w/w) albumin and saline.

According to some embodiments, the composition includes 2-10% (w/w) PVP, 12-28% (w/w) albumin and saline. According to other embodiments, the composition includes 0.1-25% (w/w) modified starch and saline.

According to some embodiments, the composition includes 1-2% (w/w) gelatin, 12-28% (w/w) albumin and phosphate buffer. According to other embodiments, the composition includes 0.5-2% (w/w) pectin and phosphate buffer. According to some embodiments, the composition includes 0.1-1% (w/w) pectin, 12-28% (w/w) albumin and phosphate buffer.

According to some embodiments, the composition includes 0.5-2% (w/w) guar gum and phosphate buffer. According to other embodiments, the composition includes 0.3-1% (w/w) pectin, 12-28% (w/w) albumin and phosphate buffer.

According to some embodiments, the composition includes 2-10% (w/w) PVP, 12-28% (w/w) albumin and phosphate buffer. According to other embodiments, the composition includes 0.1-25% (w/w) modified starch and phosphate buffer.

According to some embodiments, the composition is permeable. According to further embodiments, the composition in the solid state thereof remains permeable. The composition I the solid state thereof may include pores. According to further embodiments, the composition in the solid state has a sponge-like structure. The pores formation and size thereof may be defined by the composition ingredients and the phase transition conditions. The composition in the solid state thereof is configured to elevate the target tissue. According to further embodiments, the composition in the solid state is configured to reinforce the operated target area. The composition provides protection for the weak area of the organ after the target tissue removal. Moreover, functional groups of the injectable composition components may include hydroxyl (OH—), sulfhydryl (SH—) or amine (—NH—) groups, which have adhesive properties and may bond to the contacting tissue. The composition, which underwent phase transition is therefore configured to patch the target area after the target tissue removal. Said patching allows to increase the physician safety and prevent post operation complications, such as, but not limited to, perforation or inflammation. The controlled phase transition of the composition further prevents the injected fluid spreading over the range more than about 3-5 cm from the lifting device. According to further embodiments, the composition phase transition induces the tissue healing, such that the healing may proceed in about 4-7 days. The composition, which undergoes phase transition, is configured to dissolve in up to about 5 days, allowing essentially entire tissue regeneration prior to the dissolution. The composition phase transition forming a patch may further prevent contamination of the exposed tissue. According to additional embodiments, said patching allows increasing the cost efficiency of the target tissue removal by reducing the required hospitalization period to about 5-8 hours.

In another aspect, there is provided a device for lifting a first tissue layer with respect to a second tissue layer adjacent thereto, during an endoscopic procedure, the device comprising: an injection module having an elongate body, a proximal region and a distal region, wherein the distal region comprises at least one outlet, configured to deliver a composition configured to undergo phase transition, to a region between the first tissue layer and the second tissue layer; and a phase transition module, configured to controllably induce phase transition of the composition from the liquid state to the solid state thereof. In some embodiments, the phase transition module is enclosed in the injection module. In particular embodiments, the phase transition module is disposed in the distal region of the injection module. In other embodiments, the phase transition module is physically separated from the injection module. In some embodiments, the terms “injection module” and “injector” can be used interchangeably. In further embodiments, the terms “device” and “intruder” can be used interchangeably.

In some embodiments, the injection module is configured to deliver the composition, in the liquid state thereof to the region between the first tissue layer and the second tissue layer and the phase transition module is configured to controllably induce phase transition of the delivered composition from the liquid state to the solid state thereof. According to certain embodiments, the composition is configured to undergo phase transition outside the lifting device. Thus, in some embodiments, the lifting device provides external phase transition of the composition. In these embodiments, the device comprises: an injection module having an elongate body, a proximal region and a distal region, wherein the distal region comprises at least one outlet, configured to deliver a composition configured to undergo phase transition, in the liquid state thereof, to a region between the first tissue layer and the second tissue layer; and a phase transition module, configured to controllably induce phase transition of the delivered composition from the liquid state to the solid state thereof.

In other embodiments, the phase transition module is configured to controllably induce phase transition of the composition, disposed within the device, from the liquid state to the solid state thereof and the injection module is configured to deliver the composition, in the solid state thereof, to the region between the first tissue layer and the second tissue layer. According to certain embodiments, the composition is configured to undergo phase transition inside the lifting device. Thus, in some embodiments, the lifting device provides internal phase transition of the composition. In these embodiments, the device comprises: a phase transition module, configured to controllably induce phase transition of the composition, configured to undergo phase transition, disposed within the device from the liquid state to the solid state thereof; and an injection module having an elongate body, a proximal region and a distal region, wherein the distal region comprises at least one outlet, configured to deliver the composition in the solid state thereof, to a region between the first tissue layer and the second tissue layer. In further embodiments, the device has an elongate body, a proximal region and a distal region, wherein the proximal region comprises at least one inlet, configured to receive a composition, wherein the composition is configured to undergo phase transition from a liquid state to a solid state; the elongate body is configured to induce phase transition of the composition along the length of the elongate body; and the distal region comprises at least one outlet, configured to deliver the composition in the solid state thereof to a region between the first tissue layer and the second tissue layer.

In additional embodiments, the injection module is configured to deliver the composition, in the liquid state and/or in the solid state thereof to the region between the first tissue layer and the second tissue layer and the phase transition module is configured to controllably induce phase transition of the delivered composition and/or of the composition, disposed within the device, from the liquid state to the solid state. In some embodiments, the device is configured to deliver the composition, configured to undergo phase transition, to the region between the first tissue layer and the second tissue layer. According to certain embodiments, the composition is configured to undergo phase transition outside the lifting device and inside the lifting device. Thus, in some embodiments, the lifting device provides external and internal phase transition of the composition.

In some embodiments, the phase transition module is configured to provide heating, cooling, electromagnetic radiation or ultrasound radiation of the delivered composition. In some embodiments, the phase transition module comprises at least one electrode, connected to a power source. In some embodiments, said power source is a DC power source. In other embodiments, said power source is an AC power source. In some embodiments, the phase transition module comprises two electrodes. In further embodiments, the phase transition module comprises two or more electrodes.

In some embodiments, the phase transition module comprises bipolar electrodes, exposed at the opposite sides of the distal region of the phase transition module, wherein the exposed electrodes are configured to face the delivered composition. In particular embodiments, the phase transition module comprises two bipolar electrodes, exposed at the opposite sides of the distal region of the phase transition module, wherein the exposed electrodes are configured to face the delivered composition. In other embodiments, the phase transition module comprises concentric electrodes. In particular embodiments, the phase transition module comprises two concentric electrodes. In other embodiments, the phase transition module comprises planar electrodes. In particular embodiments, the phase transition module comprises two planar electrodes. In further particular embodiments, the concentric or planar electrodes are fully enclosed within the phase transition module. In some embodiments, the phase transition module includes a combination of the exposed bipolar electrodes and fully enclosed concentric or planar electrodes.

In some embodiments, the device further includes a cutting means disposed in the distal region of the phase transition module and/or of the injection module. A non- limiting example of the cutting means is a diathermic electrode.

The distal region of the device of the present invention may further include an orientation indicator, configured to indicate the spatial orientation of the injection module distal region. According to additional embodiments, the distal region further includes a phase transition indicator, configured to provide indication of the phase transition state of the composition. Said phase transition indicator may include a light source, such as, but not limited to, LED.

In some embodiments, the device is designed in such a way that the elongate body of the injection module comprises a sliding surface, configured to facilitate smooth sliding of the injection module upon the second tissue layer. In additional embodiments, the elongate body of the injection module comprises a bottom surface and a top surface, wherein the shape of the top surface is distinct from the shape of the bottom surface. The dissimilar shapes of the top and the bottom surfaces allow the evaluation of the spatial orientation of the device during the operation. According to yet further embodiments, the radius of the proximal region of the injection module is greater than the radius of the elongate body and the proximal region is configured to limit the injection module's travel into the area between the first tissue layer and the second tissue layer.

According to some embodiments, the injection module includes a plurality of outlets, configured to stabilize the injection module spatial orientation during the composition delivery. According to additional embodiments, the injection module further includes a mechanism, configured to extend the outlet beyond the distal region of the injection module and to retract the outlet into the distal region of the injection module.

According to further embodiments, the device for lifting a first tissue layer with respect to a second tissue layer adjacent thereto further includes a tube in a fluid-flow connection with the injection module. According to further embodiments, the tube includes at least one partition, including the composition. According to other embodiments, the tube includes at least two partitions, wherein the first partition includes the active ingredient and the second partition includes the additive. According to certain embodiments, the first partition includes the thermo-sensitive material and the second partition includes the stabilizer. According to additional embodiments, the first partition includes the thermo-sensitive material and the second partition includes the stabilizer and the color indicator. According to other embodiments, the first partition includes the chemically-active material and the second partition includes the phase-transition trigger. According to some embodiments, separating the compounds of the composition into at least two partitions improves storage and/or shelf-life of the composition. According to other embodiments, separating the compounds of the composition into at least two partitions allows conducting phase transition of the composition upon mixing of the components. According to additional embodiments, separating the compounds of the composition into at least two partitions allows defining the ratio between the compounds of the composition. In further embodiments, the tube having one or more partitions enables delivery of a homogeneous composition to the injection module.

In the preferred embodiments, the composition is delivered to the device in the liquid state thereof. Without wishing to being bound by any specific theory or mechanism of action, delivery of the liquid composition is significantly easier than delivery of a gel or a solid composition. Thus, the ability of the device of the present invention to receive the composition in the liquid state thereof and deliver to the target region in the solid state is an additional advantageous feature of the present invention.

According to some embodiments, the device further includes an actuator, configured to assist the delivery of the composition, in the solid state thereof, to the region between the first tissue layer and the second tissue layer. According to a certain embodiment, the actuator includes a piston concentrically configured with the electrodes, and a spring, associated with the piston.

According to some embodiments, the device further includes a dosing module, configured to be in a fluid-flow connection with the proximal region of the device and further configured to provide a metered delivery of the composition in the solid state thereof, to the region between the first tissue layer and the second tissue layer. According to a certain embodiment, the dosing module includes a ratchet mechanism.

According to some embodiments, the device of the present invention is configured to be operated through an endoscope. According to further embodiments, the device can be used in a gastrointestinal endoscopic procedure. According to a certain embodiment, the endoscopic procedure is endomucosal resection (EMR) and/or endoscopic submucosal dissection (ESD). The endomucosal resection can be an en- bloc EMR or a piecemeal EMR. According to additional embodiments, the endoscopic procedure is performed on a mammalian subject.

In another aspect, there is provided an endoscopic system including an endoscope and the device for lifting a first tissue layer with respect to a second tissue layer adjacent thereto, wherein the device is operable through the endoscope. According to some embodiments, the endoscopic system further includes a control unit. According to further embodiments, the control unit includes a user interface, configured to provide real time information and/or control of the device position and spatial orientation. According to additional embodiments, the user interface includes virtual tools configured to provide real time information and/or control of the device position and spatial orientation. According to further embodiments, the user interface is further configured to provide real time information and/or control of the an extent of the phase transition of the composition, a proportion of the composition which undergoes phase transition, a position of the composition which undergoes phase transition, a rate of the phase transition of the composition, a duration of the phase transition of the composition or any combination thereof. Each possibility represents a separate embodiment of the invention. According to particular embodiments, the user interface provides real time information and/or control of the phase transition of the composition which proceeds within the region between the first tissue layer and the second tissue layer.

In still another aspect, there is provided an endoscopic system including an endoscope and a control unit, including a user interface, configured to provide real time information and/or control of the endoscope position and spatial orientation. According to additional embodiments, the system may further include the device for lifting a first tissue layer with respect to a second tissue layer adjacent thereto. In yet another aspect there is provided a method for lifting a first tissue layer with respect to a second tissue layer adjacent thereto, during an endoscopic procedure, the method including: inserting the device between the first tissue layer and the second tissue layer; delivering, using the injection module, a composition configured to undergo phase transition, to a region between the first tissue layer and the second tissue layer; and controllably inducing, using the injection module, the phase transition of the composition configured to undergo phase transition from the liquid state to the solid state thereof. In some embodiments, the step of delivering of the composition precedes the step of controllably inducing the phase transition of the composition. In other embodiments, the step of delivering of the composition follows the step of controllably inducing the phase transition of the composition.

According to some embodiments, the injection module is inserted into the distal end of region between the first tissue layer and the second tissue layer. According to further embodiments, the method further includes: backward sliding of the injection module towards the insertion point; and repeating the step of inducing phase transition of the composition. According to some embodiments, the step of inducing phase transition of the composition are repeated until the substantial elevation of the first tissue layer with respect to the second tissue layer is achieved.

According to further embodiments, the method further includes: backward sliding of the device towards the insertion point; repeating the step of inducing phase transition of the composition; and repeating the step of delivering the composition. According to some embodiments, the step of inducing phase transition of the composition and the step of delivering the composition are repeated until the substantial elevation of the first tissue layer with respect to the second tissue layer is achieved.

According to further embodiments, the method includes repetitions of the steps of delivering the composition and/or controllably inducing phase transition thereof. In some embodiments, the steps are repeated following a prolonged period of time. Without wishing to being bund by any specific theory or mechanism of action, following the initial phase transition of the composition, the region between the first tissue layer and the second tissue layer is secured with a patch, thus allowing the physician to stop the endoscopic procedure and renew his work up to 8-12 hours following the previously performed steps of the composition delivery and controllable phase transition induction.

In some embodiments, the method is used during an endoscopic gastrointestinal procedure, selected from en-bloc EMR, piecemeal-EMR or ESD. In some embodiments, the physician can switch between the procedures, while the method of the present invention provides the prolonged lifting of the first tissue layer with respect to the second tissue layer.

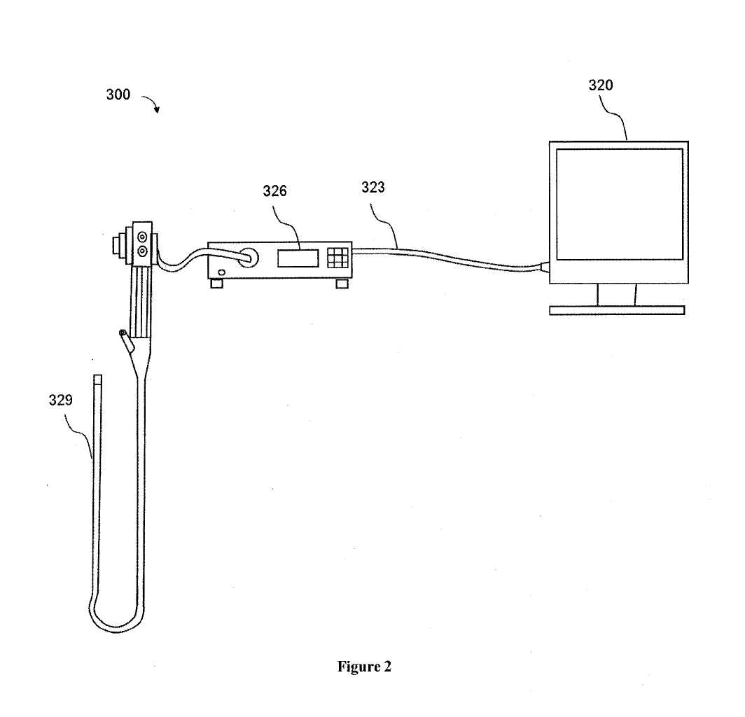

Reference is now made to FIG. 2, which schematically illustrates endoscopic system 300, according to some embodiments of the invention. Endoscopic system 300 includes display 320, Surgical Control Unit (SCU) 326 and endoscope 329. Display 320 is connected to SCU with cable 323. Cable 323 is configured to transmit video signal. SCU 326 is configured to provide image processing. According to some embodiments, SCU 326 is configured to provide image processing in real-time. SCU 326 includes image processing software, providing the user with operation guiding lines, indicating a safe operational region.

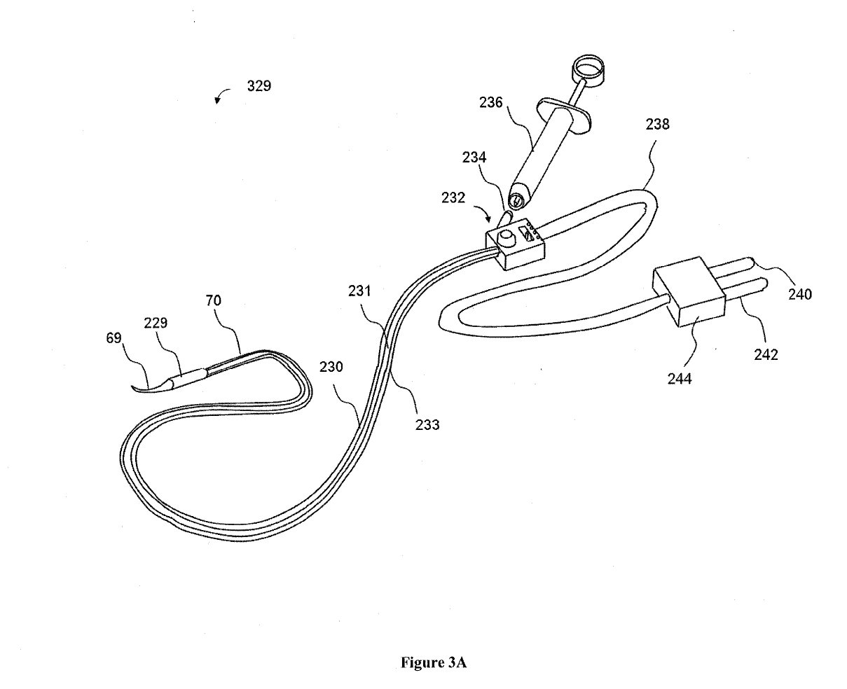

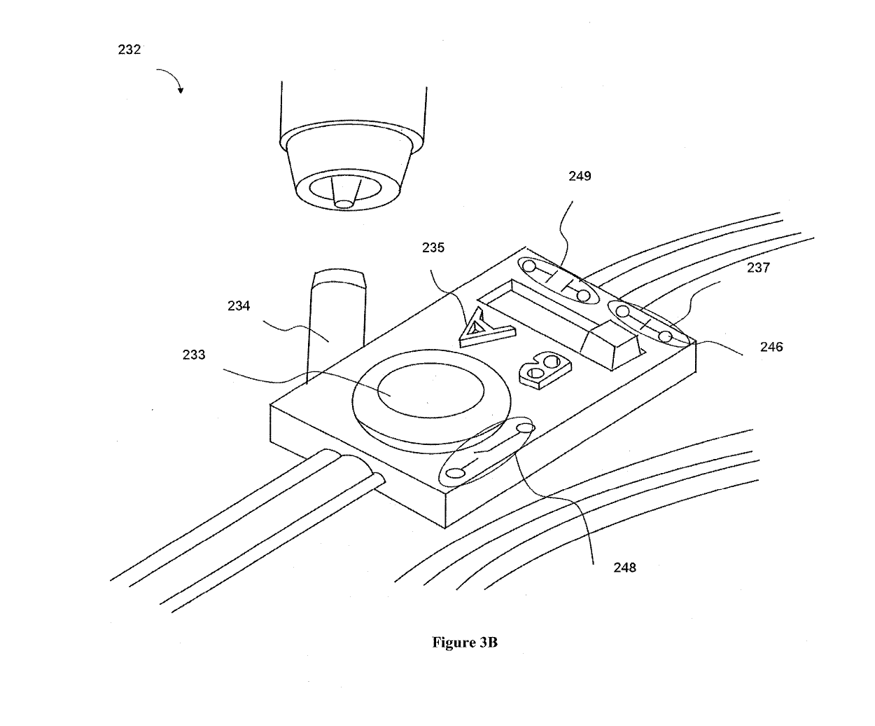

Reference is now made to FIG. 3A, which schematically illustrates endoscope 329, according to some embodiments of the invention. Endoscope includes power supply plug 244, including pins 240 and 244, control box 232, intruder 69 and tube 70. According to some embodiments, intruder 69 is a separate unit. According to other embodiments, intruder 69 is integrated with tube 70 as one structure. Tube 70 is in a fluid-flow connection with intruder 70. Tube 70 is connected to intruder 69 through tube adaptor ring 229. The length of tube 70 corresponds to the length of intruder 69. According to some embodiments, tube 70 is flexible. According to other embodiments, tube 70 is rigid. Intruder 69 includes one or more openings (not shown) for transferring the injectable composition into or under the target tissue. Intruder 69 and tube 70 are in fluid-flow connection with control box 232. Intruder 69 and tube 70 are connected to control box 232 through utilities tube 231, configured to transfer the injectable composition from control box 232 to intruder 69 through tube 70, and through utilities cables 230 and 233, configured to provide electrical connection of intruder 69 to control box 232 and to the electricity source. Control box 232 includes a chamber, optionally constructed as a multi chamber, configured to introduce the injectable composition into utilities tube 231. According to some embodiments, separate chambers contain different components of the injectable composition. Control box 232 includes fast connector 234. According to some embodiments, connector 234 is a fast connector. According to specific embodiments, connector 234 is a Lure type connector. Connector 234 is configured to allow transferring the injectable composition from storage injector 236 to control box 232, wherein storage injector 236 may be connected to control box 232 through connector 234. Control box 232 is further connected to power supply plug 244 through electrical cable 238. Electrical cable 238 is configured to electrically connect control box 232 and intruder 69 to the electrical power source. According to some embodiments, power supply plug 244 is configured to be connected to RF power supply. According to other embodiments, power supply plug 244 can be connected to AC/DC power source. According to additional embodiments, power supply plug includes an RF power amplifier.

Reference is now made to FIG. 3B, which schematically illustrates a detailed view of control box 232, according to some embodiments of the invention.

Control box 232 is configured to provide connection to storage injector 236 through connector 234. Control box 232 includes bush button 233, configured to switch on and/or switch off the bipolar electrode applicator (not shown). Control box 232 further includes mode switch 237, configured to allow switching between various modes of the bipolar electrode applicator operation and mode indicator 235, configured to present the current operation mode of the bipolar electrode applicator.

Control box 232 further includes mode selector controls 248 and 249, configured to prevent a double control of the bipolar electrode by the external control unit (not shown) and by control box 232. If the bipolar electrode applicator operation mode is set to be controlled by the external control unit, mode selector controls 248 and 249 disable mode switch 237 operation.

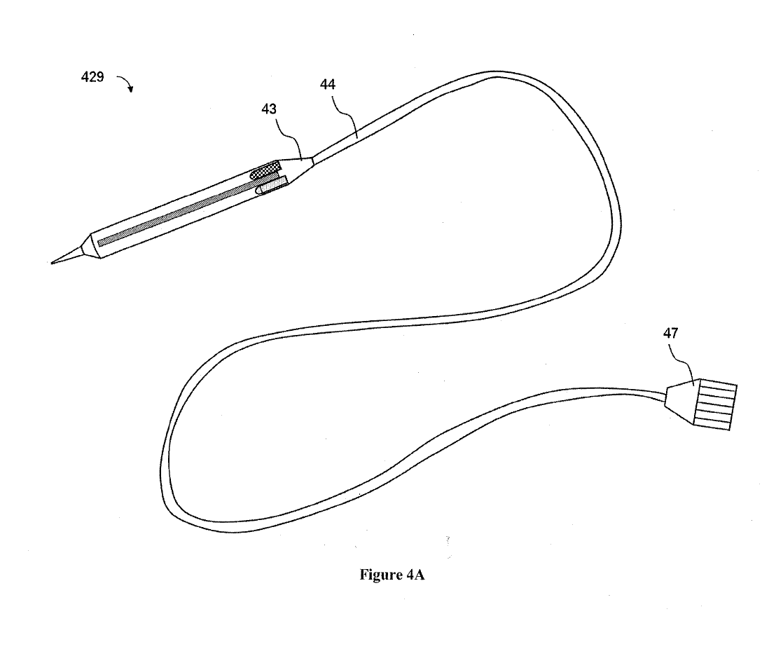

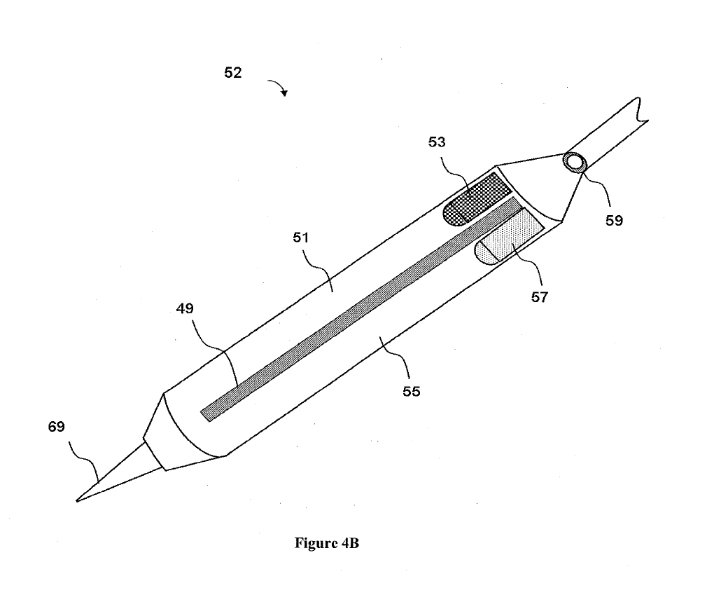

Reference is now made to FIG. 4A, which schematically illustrates endoscope 429, according to some embodiments of the invention. Endoscope includes power supply plug 47, intruder 69 and integral composition container 52, connected to intruder 69. Intruder 69 and integral composition container 52 include sliding surface 43, configured to facilitate backward motion of intruder 69 during the injection process. According to some embodiments, intruder 69 is a separate unit. According to other embodiments, intruder 69 is integrated with integral composition container 52 as one structure. Integral composition container 52 is connected to plug 47 through utility tube 44. According to some embodiments, intruder 69 is configured to deliver the chemically-active material and the phase transition trigger to the target area. According to other embodiments, intruder 69 is configured to deliver the thermally-sensitive material to the target area. According to other embodiments, intruder 69 is configured to deliver the thermally-sensitive material and the additive to the target area.

Reference is now made to FIG. 4B, which schematically illustrates a detailed view of integral composition container 52, according to some embodiments of the invention. Integral composition container 52 is connected at the distal region thereof to intruder 69. Container 52 includes chambers 51 and 55. According to some embodiments, chamber 51 contains the active ingredient and chamber 55 contains the additive. According to other embodiments, chamber 55 contains the active ingredient and chamber 51 contains the additive. According to some embodiments, chamber 51 contains the chemically-active material and chamber 55 contains the phase-transition trigger. According to other embodiments, chamber 55 contains the chemically-active material and chamber 51 contains the phase-transition trigger. According to some embodiments, chamber 51 contains the thermo-sensitive material and chamber 55 contains the stabilizer. According to other embodiments, chamber 55 contains the thermo-sensitive material and chamber 51 contains the stabilizer.

According to some embodiments, chambers 51 and 55 volumes are similar According to other embodiments, the volumes are different. Chambers 51 and 55 include pistons 53 and 57, respectively, which are disposed at the proximal region of integral composition container 52. Pistons 53 and 57 are configured to propel the comprised compositions towards intruder 69. Container 52 further includes mixing area 49, configured to facilitate mixing of the compositions comprised in chambers 51 and 55, prior to entering intruder 69. Integral composition container 52 further includes connection area 59, including a Lure connector and a non-return valve (not shown), configured to connect the proximal region of the container with utilities tube 44 (shown in FIG. 4A hereinabove).

Chambers 51 and 55 contain a sufficient volume of the composition, configured to induce pistons 53 and 57 operation. According to some embodiments, the additive is configured to act as a mediator and transfer the motion from the distal region of container 52 to piston 53 or 57, during the injection procedure. According to other embodiments, the additive is configured to dilute the active ingredient and to reduce the active ingredient volume to the minimal volume required to elevate the target tissue.

According to some embodiments the compositions comprised in chambers 51 and 55 can be in a liquid, powder or granular form. Each possibility represents a separate embodiment of the invention.

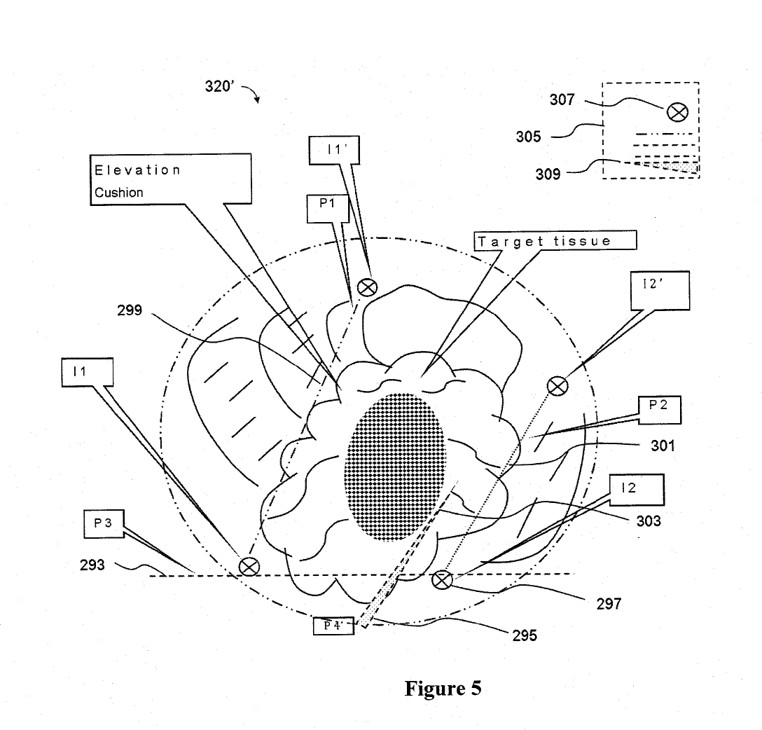

Reference is now made to FIG. 5, which schematically illustrates display 320 output 320′, according to some embodiments of the invention. Display output 320′ includes an image captured by endoscope 329 and transmitted from endoscope 329 through Surgical Control Unit 326 and cable 323 to display 320 (shown in FIG. 2) and visual tools. The purpose of the visual tools is to give the user dynamic indication of endoscope 329 steering. All lines are presented dynamically on the endoscope display and are designed to increase the orientation of the user regarding the intruder's location under the tissue during the elevation section of the operation. The visual tools include auxiliary lines, such as minimum secure elevation line 293, configured to indicate a minimal height of a lesion, which is safe for performing an operation and target tissue border lines 299 and 301, configured to indicate an allowable operation area below the target tissue. The setup procedure for marking lines such as lines 293, 299 and 301 includes pointing at the endoscope image that appears on display 320. The lines assist the surgeon to keep the intruder in the pre-planned area of the operation. Moreover, the user may at any time, measure distances on the screen such as level of elevation above the organ walls, and to verify that it is above the safe range of 8-12 mm, which is required in order to perform a safe removal of the target tissue. Auxiliary lines 293, 299 and 301 are presented with visible colors on display 320 during the procedure and move according to endoscope 329 movement. The visual tools further include intruder vector marker 295, configured to indicate the endoscope intruder position and orientation in three-dimensional environment and intruder tip marker, configured to indicate the endoscope intruder tip position in three-dimensional environment. Intruder vector marking 295 may be created by marking the start point which indicates the end of the working channel on display 320 and marking the second point on the intruder tip prior to introducing it to the organ. Use of intruder vector marking 295 allows improving the safety of the operation, reducing the duration of the procedure, and simplifying endoscope system 300 operation. Intruder propagation in the vicinity of the target tissue can be tracked by image processing of the LED light and/or by a solid state gyroscope located at the intruder tip and calibrated with the system image processor. According to some embodiments, visual tools are user controllable. User controllable working tools may include marking point 297, configured to select specific point on display output 320′ or an area defined by a plurality of marking points 297. Marking point 297, intruder vector marker 295 and auxiliary lines 293, 299 and 301 are represented by icons marking point icon 307, intruder vector marker icon 309 and auxiliary line icon 310, respectively. Icons 307, 309 and 310 are displayed in tools storage toolbox 305 on display output 320′.

According to some embodiments, the visual tools are configured to assist the user to pre-plan the operation and/or allow the user to avoid unnecessary actions during the operation. Use of said visual tools further improves safety and increases the user confidence in endoscope system 300.

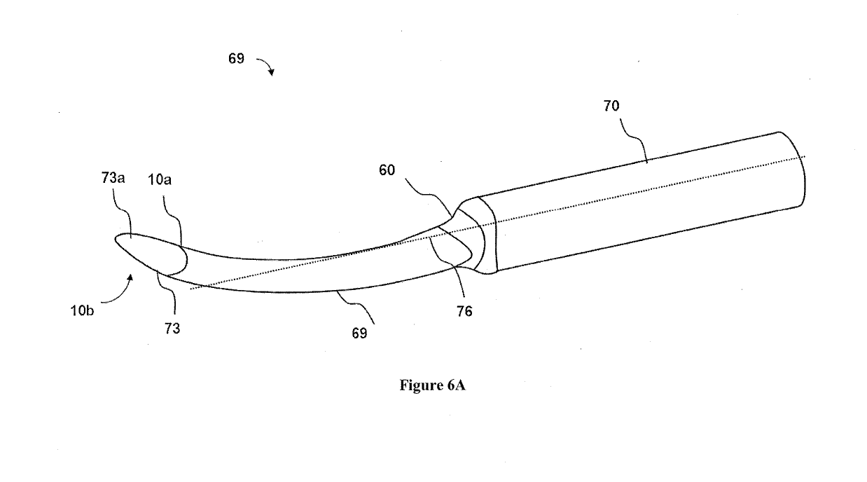

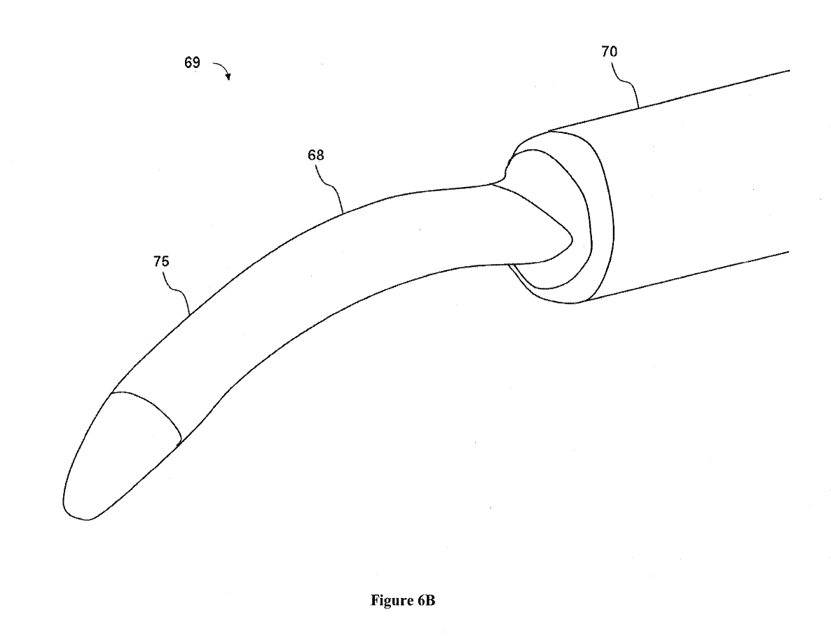

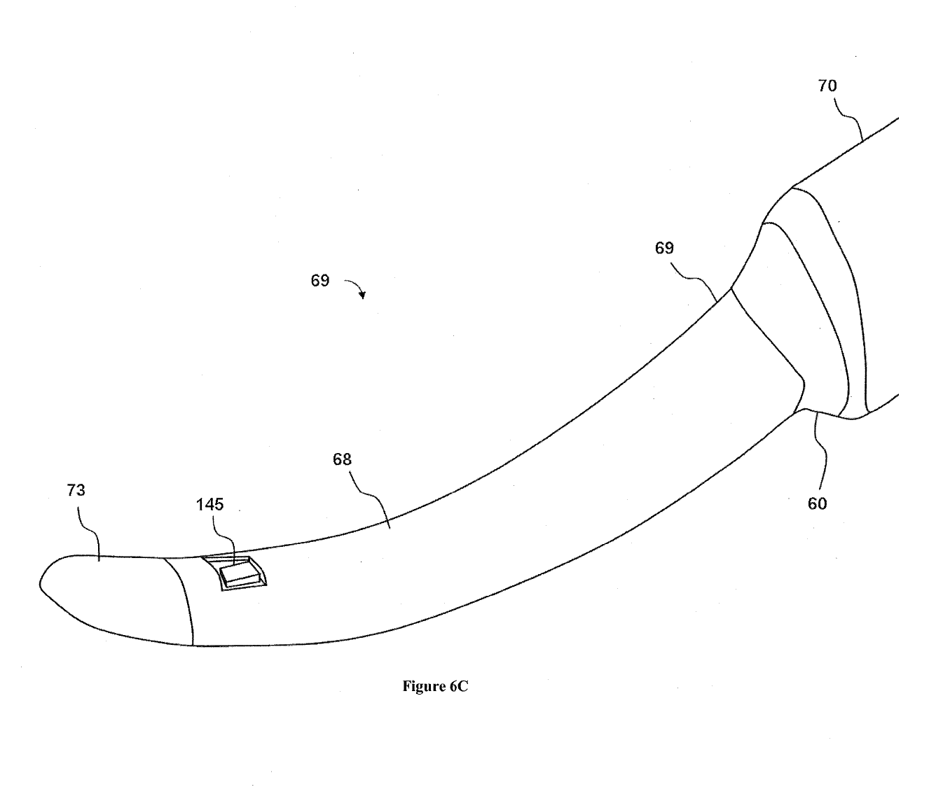

Reference is now made to FIG. 6A, which schematically illustrates intruder 69, connected to tube 70, according to some embodiments of the invention. Intruder 69 includes proximal end 60, connected to tube 70 and distal end 73, including intruder tip 74, configured to provide injection of the injectable composition. Intruder 69 further comprises an elongated shape, stretched along longitudinal axis, denoted as 76. Distal end 73 includes upper surface 10a and bottom surface 10b. According to some embodiments, surface 10a has a different shape than surface 10b, in order to allow a user to evaluate a spatial orientation of the intruder during the insertion and injection process. Intruder 69 further includes body 68, configured to contact body tissue. According to some embodiments, intruder distal end 73 has an oval shape. According to further embodiments, intruder 69 is bent, with its proximal end 60 and its distal end 73 being elevated above intruder body 68. According to an exemplary embodiment, intruder 69 is 7-10 mm long and 1-1.3 mm wide. According to further exemplary embodiments, intruder's distal end 73 is being elevated above intruder body 68 by about 0.7-0.1 mm The intruder is inserted under the target tissue with distal end 73 pointing upwards, such that intruder tip 73a is elevated above intruder proximal end 60 about 1.8-2.3 mm That shape and configuration simplicities the insertion of intruder 69 under the target tissue and maintains intruder tip 73a in a safe distance from the underlying layer of the target tissue. According to further embodiments, proximal end 60 has a stepped structure, configured to contact the elevated tissue without applying additional forces to the tissue. Proximal end 60 is further configured to stop intruder 69 backward motion during the procedure and to limit the protrusion of intruder 69, when required, for example, upon contacting target tissue boundary with proximal end 60 of intruder 69. According to exemplary embodiments, proximal end 60 stepped shape dimensions are in a range of about 1.5-3.2 mm. According to some embodiments, intruder 69 is made from ceramic material, polymeric material or a combination thereof.

Reference is now made to FIG. 6B, which schematically illustrates intruder 69, connected to tube 70, wherein intruder 69 is shown in its upturned position, according to some embodiments of the invention. The bottom side of intruder body 68 includes sliding surface 75, configured to provide sliding of intruder 69 upon contacting the underlying layer of the target tissue. According to some embodiments, sliding surface 175 is configured to induce backward sliding of intruder 69 during the injection process.

Reference is now made to FIG. 6C, which schematically illustrates intruder 69, connected to tube 70, wherein intruder 69 includes inspection LED 145. Inspection LED 145 is disposed on intruder body 68, close to intruder distal end 73 and is configured to allow control over of the phase transition process. According to exemplary embodiments, LED 145 is disposed about 0-2 mm from intruder tip 73a. LED 145 wavelength spectrum may be in the range of about 450-570 nm LED 145 radiation may be observed by the user via the endoscope's imager (not shown). Phase transition from liquid to solid state increases turbidity of proteins from about 5-50 NTU to about 400-500 NTU. The phase transition can therefore be evaluated by inspecting LED radiation. Accordingly, LED 145 allows controlling the phase transition process, determining the state of the injected composition phase transition in real time and executing phase transition process according to the instant state of the injected substance.

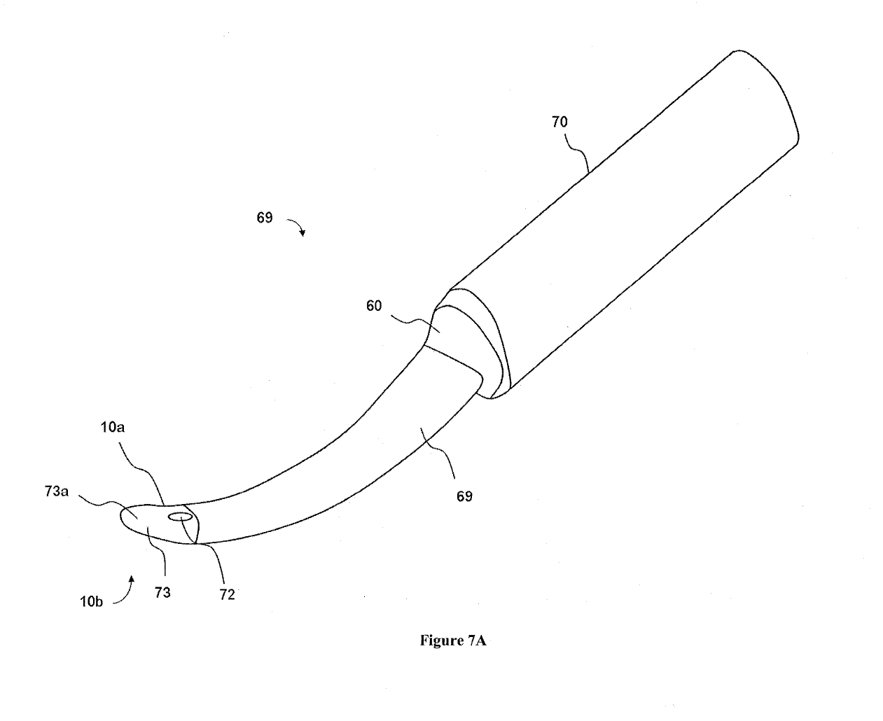

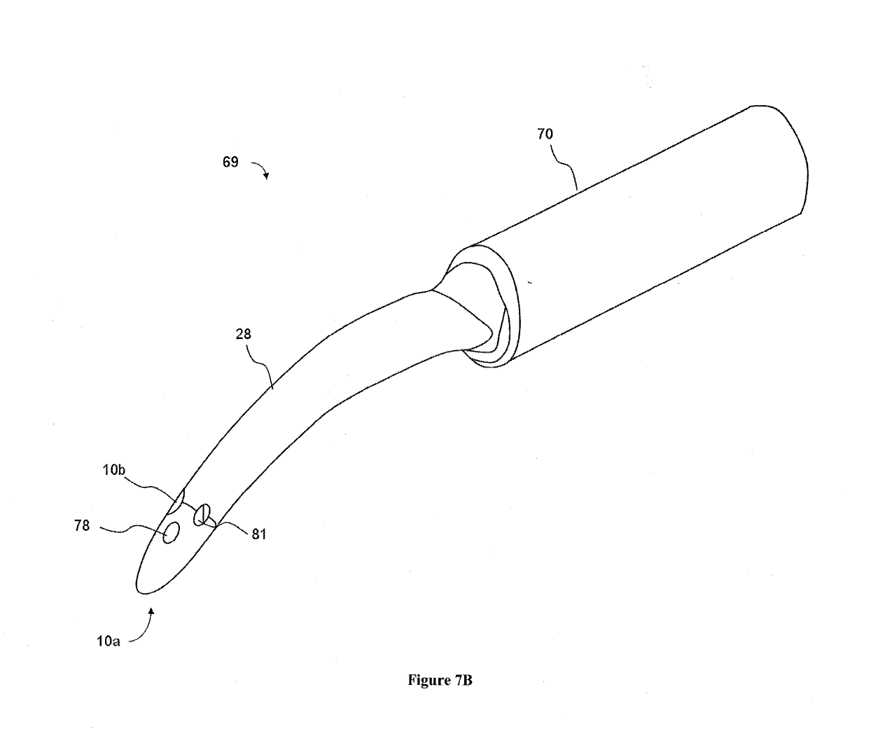

Reference is now made to FIG. 7A, which schematically illustrates intruder 69, connected to tube 70, wherein intruder 69 includes injection outlets and to FIG. 7B, which schematically illustrates intruder 69, connected to tube 70, wherein intruder 69 includes injection outlets and wherein intruder 69 is shown in its upturned position, according to exemplary embodiments of the invention. Intruder 69 includes proximal end 60, connected to tube 70, body 68 and distal end 73, configured to provide injection of the injectable composition, as presented in FIG. 6A hereinabove. Distal end 73 includes upper surface 10a and bottom surface 10b. Intruder 69 further includes a plurality of injection outlets, such as injection outlets 72, 78 and 81. According to some embodiments, distal end bottom surface 10b includes 2-5 injection outlets, such as outlets 78 and 81. According to other embodiments, distal end top surface 10a includes about 1-2 injection outlets, such as outlet 72. According to some embodiments, injection outlet 78 is a central injection outlet, configured to provide injection of the injectable composition into the target tissue. According to additional embodiments, injection outlets 81 are non-central outlets, configured to increase the injection area of the injectable composition and to improve injection efficiency. According to exemplary embodiments, injection outlets 72 are disposed about 0.4-0.6 mm from intruder 69 tip 73a.

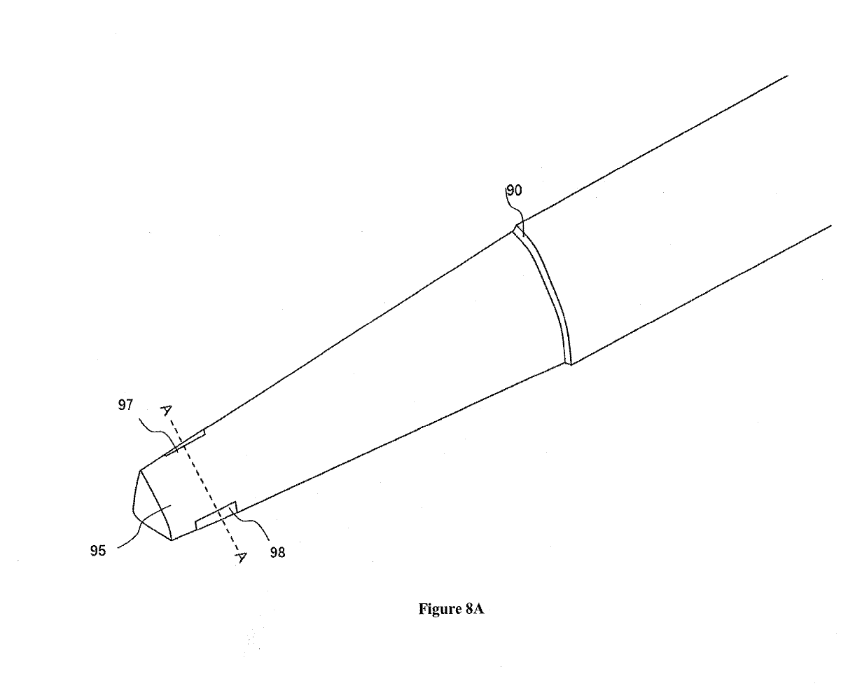

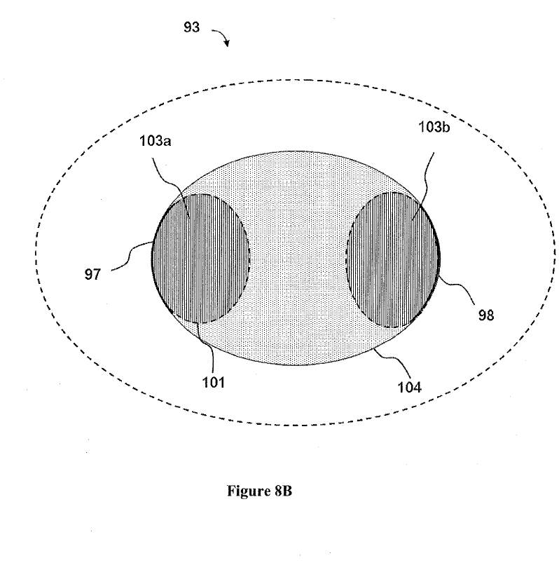

Reference is now made to FIG. 8A, which schematically illustrates heating module 93, and to FIG. 8B, which schematically illustrates a cross-section view of heating module 93, along line AA in FIG. 8A, according to some embodiments of the invention. Heating module 93 is a non-bended bipolar heating module, configured to provide phase transition of the injectable composition. Heating module 93 is configured to be operated concurrently with intruder 69, presented in FIGS. 7A and 7B. Heating module 93 includes proximal end 90, distal end 95 and heating module body 92. Proximal end 90 is covered by a protective insulation coating. Distal end 95 includes protective insulation coating 101 and 104, and clear sections 97 and 98, devoid of protective insulation. Distal end 95 further includes exposed electrodes 103a and 103b. Electrodes 103a and 103b are disposed inside heating module 93 and are exposed by clear sections 97 and 98. Electrodes 103a and 103b are configured to heat the injectable composition and to induce the phase transition thereof. According to some embodiments, electrodes 103a and 103b are of similar size, wherein electrode 100 is connected to a first pole of the power supply (not shown) and electrode 103 is connected to a second pole of the power supply. Electrodes 103a and 103b are disposed on two opposing sides of distal edge 95 and are oriented such that the first side of electrode 103a faces the injected solution, surrounding heating module 93 and the second side of electrode 103a faces the first side of electrode 103b; and the first side of electrode 103b faces the second side of electrode 103a and the second side of electrode 103b faces the injected solution. Electrodes 103a and 103b are further configured to prevent adherence of the injectable composition, which underwent phase transition. According to some embodiments, electrodes 103a and 103b surfaces, contacting insulation coating 101 and 104, contain micro-capillaries (not shown) that are connected to an external module operated through the endoscope's working channel. Said micro-capillaries are configured to further prevent sticking of the injectable composition, which underwent phase transition, to electrodes 103a and 103b. Said micro-capillaries may be filled with pressurized gas, such as, but not limited to, air. Said micro-capillaries further include nozzles, from which said gas is ejected, induced by pneumatic system of the external module.

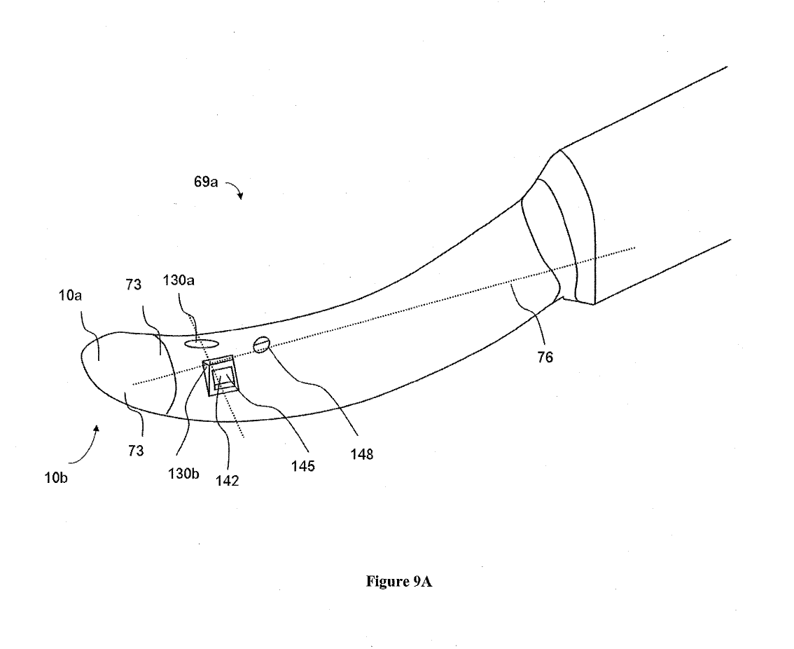

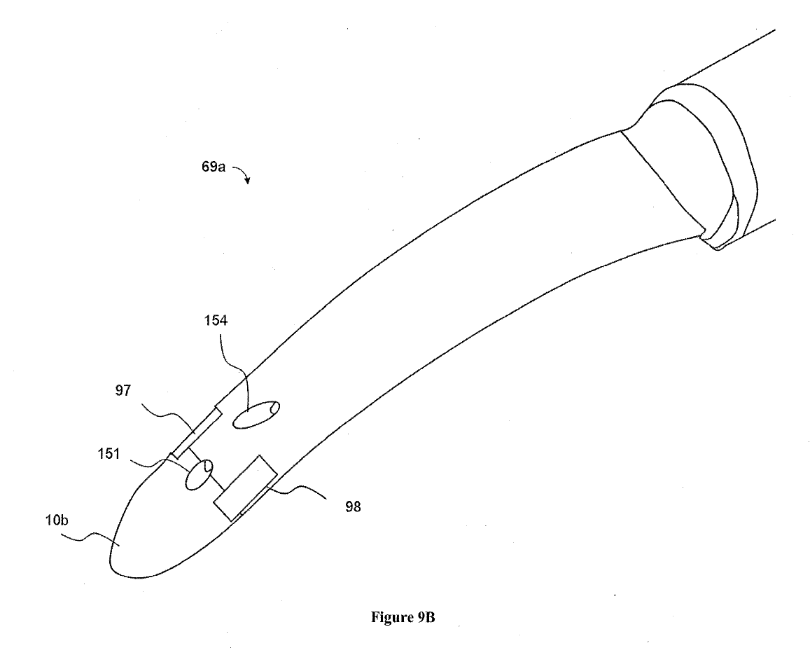

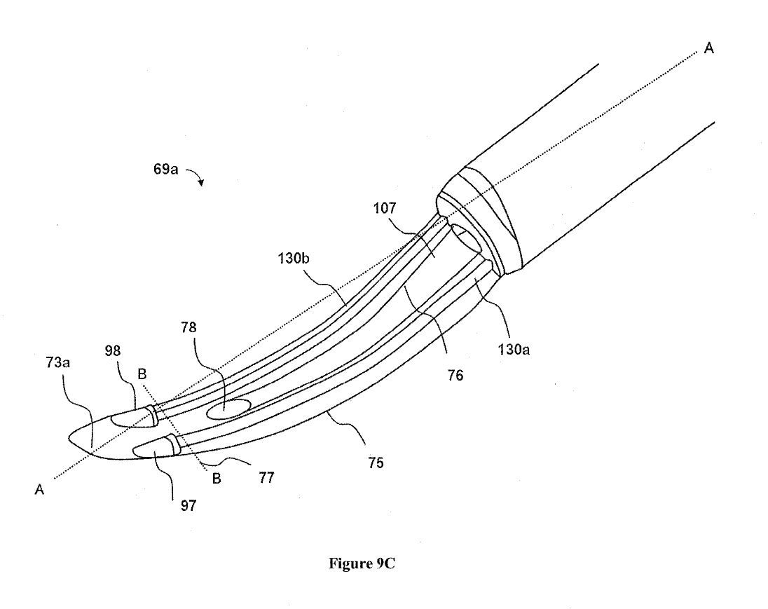

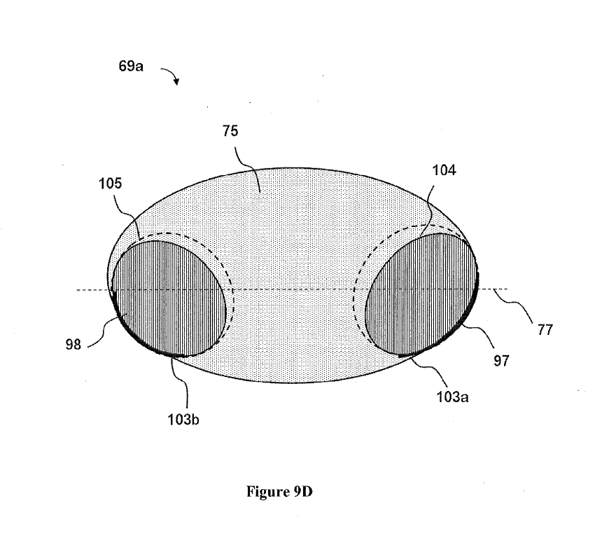

Reference is now made to FIG. 9A, which schematically illustrates integrated intruder 69a, combining intruder 69 (presented in FIGS. 7A and 7B) and heating module 93 (presented in FIG. 8A and 8B) and to FIG. 9B, which schematically illustrates integrated intruder 69a in its upturned position. Integrated intruder 69 includes a plurality of injection outlets, such as injection outlets 130a, 130b, 148, 151 and 154. Injection outlets 130a, 130b and 148 are disposed on top surface 10a of distal end 73 of integrated intruder 69a. The injection outlets may be disposed in specific positions, allowing stabilization of integrated intruder 69a during the injection process. According to some embodiments, injection outlets 130a, 130b and 148 are stabilizing outlets. According to further embodiments, injection outlets 130a and 130b are disposed at a similar distance from longitudinal axis 76, and at a similar degree with respect to said axis. Injection outlet 148 is disposed along longitudinal axis 76, at a specific distance from injection outlets 130a and 130b. Such orientation allows mutual counterpoising of the injection jets, exiting from injection outlets 130a, 130b and 148 and prevents integrated intruder 69a instability, such as trembling and/or wobbling during the injection process.

Additional injection outlets are disposed on bottom side 10b of distal end 73 of integrated intruder 69a, such as injection outlets 151 and 154 (as presented in FIG. 9B). According to some embodiments, injection outlet 151 is a central outlet and is configured to deliver the major portion of the injectable composition to the target area. According to additional embodiments, injection outlet 154 is a stabilizing outlet and is configured to assist balancing of integrated intruder 69, similarly to the injection outlets 130a, 130b and 148. According to further embodiments, injection outlets 151 and 154 are disposed along longitudinal axis 76.

On the bottom side 10b integrated intruder 69a further includes clear sections 97 and 98, configured to expose electrodes, disposed inside integrated intruder 69 (as presented in FIG. 9C hereinbelow). Clear sections 97 and 98 allow the injected fluid contact with the electrodes, inducing phase transition of the injected fluid.

Integrated intruder 69a further includes intruder inspection LED 145 disposed in LED housing 142 (as presented in FIG. 9A). LED 145 is disposed at a specific distance from longitudinal axis 76 and from clear sections 97 and 98. According to further embodiments, LED housing 142 walls are chamfered, in order to form homogenous lamination of elevated tissue during the denaturation inspection.

Reference is now made to FIG. 9C, which schematically illustrates integrated intruder 69a internal view, according to exemplary embodiments of the invention. Integrated intruder 69a further includes injection channel 107, configured to transfer a flow of the injectable composition to the injection outlets, such as outlet 151. Integrated intruder 69a further includes bipolar electrodes 103a and 103b, disposed along longitudinal axis 76, at opposite sides of integrated intruder 69. Electrodes 103a and 103b are almost entirely isolated by integrated intruder casing 75 (only partially shown in FIG. 9C), wherein only a small portion of electrode 130a surface is exposed through clear section 97 and a small portion of electrode 130b surface is exposed through clear section 98. Isolation of external portion of electrodes 103a and 103b by casing 75 allows focusing electromagnetic field to specific points along integrated intruder 69a. Clear sections 97 and 98 are disposed in the proximity of integrated intruder tip 73a, allowing contact of electrodes with the injected fluid in the vicinity of integrated intruder tip 69. According to a certain embodiment, clear sections 97 and 98, configured to expose electrodes 103a and 103b, respectively, are disposed at a distance of about 1-3 mm from integrated intruder tip 73a on the sliding surface of integrated intruder 69a at an angle of about 0-5 degrees with respect to the lateral axis of integrated intruder distal end 73, denoted as 77.

Reference is now made to FIG. 9D, which schematically illustrates a cross-section view of integrated intruder 69a, along line AA in FIG. 9A, according to some embodiments of the invention. Cross-section of integrated intruder 69a along line AA includes protective coating 104 and 105, configured to isolate an internal portion of electrodes 103a and 103b, to prevent the injectable composition phase transition inside integrated intruder 69a, upon application of voltage to electrodes 103a and 103b. Cross-section further presents clear sections 97 and 98, contacting protective coatings 104 and 105, respectively, wherein clear sections 97 and 98 are configured to expose a small portion of electrodes 103a and 103b, facing the injected fluid, surrounding intruder 69a tip, as explained hereinabove. Electromagnetic field is generated between electrodes 103a and 103b upon application of voltage to the electrodes, wherein the electric contact between the electrodes is established by the charge transfer inside the injectable composition, transferred to the target area prior or/and during voltage application to the electrodes. Electrical resistance of the injectable composition depends on the distance between the electrodes contacting the injectable composition, such that altering the position of clear windows 97 and 97 with respect to lateral axis 77 of integrated intruder distal end 73 and/or to each other allows increasing or decreasing electric field amplitude and focusing electric field at specific areas in the injected fluid. In embodiments, where clear sections are positioned at bottom surface 10b of integrated intruder 69a distal end, the electromagnetic field is higher in the vicinity of bottom surface 10b than in the vicinity of top surface 10a of integrated intruder 69a distal end. In these embodiments, application of voltage to electrodes 103a and 103b provides heating of the injected fluid, which is located below integrated intruder tip 70, wherein the heating of the area below integrated intruder 69a is more intense, relatively to the area above integrated intruder 69a, therefore allowing controllable and/or selective phase transition of the injected fluid.

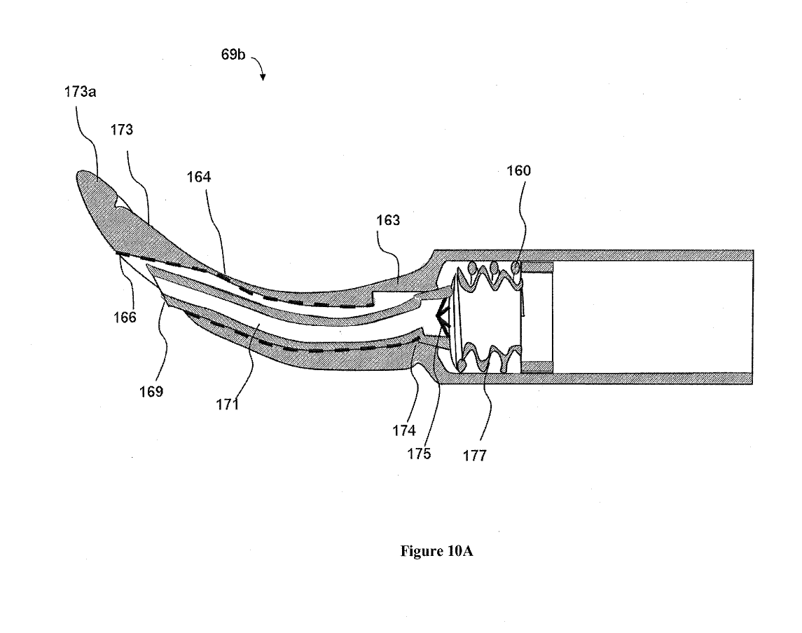

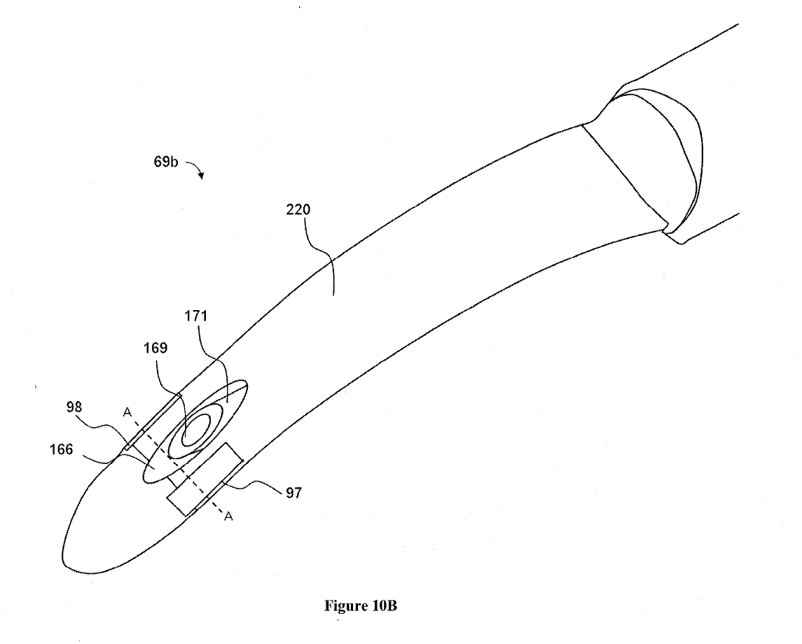

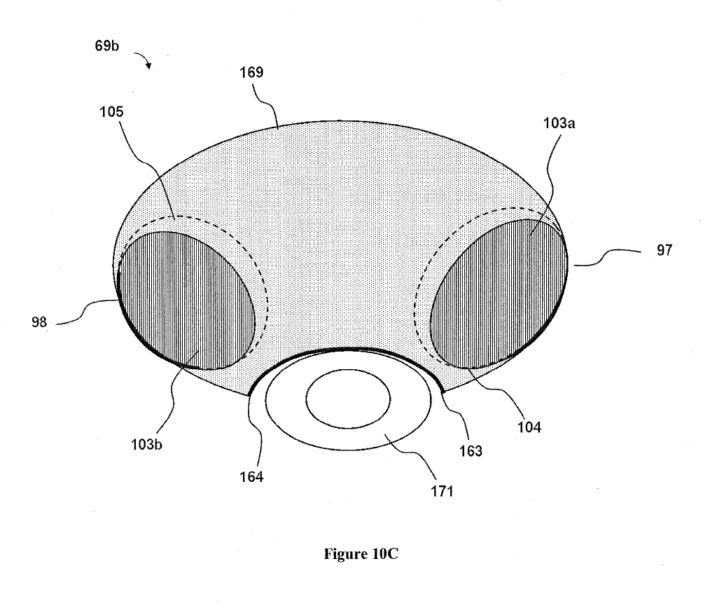

Reference is now made to FIG. 10A, which schematically illustrates a cross section of integrated retractable intruder 69b. Integrated retractable intruder 69b is a variation of integrated intruder 69a, including a retractable injector mechanism. A retractable injector mechanism includes injection channel 163, retractable injector 171, piston 175 and spring 160. Retractable injector 171 is disposed inside injection channel 163. Retractable injector 171 allows delivering the injectable composition through the front side thereof, to the target area located below integrated intruder tip 173a, when retractable injector 171 is in retracted position and delivering the injectable composition to the target area located beyond integrated intruder tip 70, wherein tip 70 does not contact and/or reach said target area, when retractable injector 171 is in extended position. The front side of retractable injector 171 includes distal end 166, contacting distal end 69b of injection channel 163. According to some embodiments, distal end 173 of retractable injector 171 is disposed about 0.2-0 5 mm from the casing of integrated retractable intruder 69b, in order to prevent contact between the target tissue and retractable injector 171 during the introduction of integrated retractable intruder 69b to the target area. The rear side of retractable injector 171 contacts piston 175. Piston 175 is configured to facilitate retractable injector extension beyond integrated intruder tip 173a and spring 160 is configured to facilitate retractable piston retraction back into retractable intruder 69b. According to some embodiments, retractable intruder 69b further includes expandable tube 177, contacting spring 160, piston 175 and being associated with retractable injector 171. Expandable tube 177 is configured to expand upon the extension of retractable injector 171 and to compress upon the retraction thereof. Expandable tube 177, therefore, is configured to assist retractable injector 171 extension and retraction. Expandable tube is further configured to provide sealing of the injectable composition filling retractable injector 171.

According to further embodiments, the retractable injector mechanism includes stopper 174, configured to define the maximal protrusion of retractable injector 171. The stroke of retractable injector 171 is, therefore, defined by the position of stopper 174 and by an extent of expandable tube 177 compression. Stopper 174 may include elastic sealers, such as, but not limited to, o-rings, wipers or rings with fins, configured to seal against the walls of injection channel 163, comprising retractable injector 171 to prevent leakage of the injectable composition.

According to still further embodiments, injection channel 163 includes anti-friction wall coating 164, configured to prevent the leakage of the injectable composition from retractable injector 171 and to reduce the friction of retractable injector 171 motion during extension and retraction thereof.

Reference is now made to FIG. 10B, which schematically illustrates integrated retractable intruder 69b in its upturned position, according to some embodiments of the invention. Sliding surface 220 of retractable intruder 69b includes injection channel distal end 166 and retractable injector distal end 169. Retractable injector 171 is configured to extend from retractable intruder 69b through injection channel distal end 166 opening. Sliding surface 220 further includes clear sections 97 and 98, exposing electrodes 103a and 103b (shown in FIG. 10C hereinbelow).

Reference is now made to FIG. 10C, which schematically illustrates a cross-section view of integrated retractable intruder 69b, along line AA in FIG. 10B, wherein the cross-section is depicted in the upturned position relative to FIG. 10B, according to some embodiments of the invention. Cross-section of retractable intruder 69b along line AA includes protective coating 104 and 105, configured to isolate an internal portion of electrodes 103a and 103b, to prevent the injectable composition phase transition inside integrated retractable intruder 69b, upon application of voltage to electrodes 103a and 103b. Cross-section further presents clear sections 97 and 98, contacting protective coatings 104 and 105, respectively, wherein clear sections 97 and 98 are configured to expose a small external portion of electrodes 103a and 103b, as explained hereinabove. The cross-section of integrated retractable intruder 69b along line AA further includes retractable injector 171, protruding from injection channel 163, wherein the walls of injection channel 163 are coated with anti-friction coating 164.

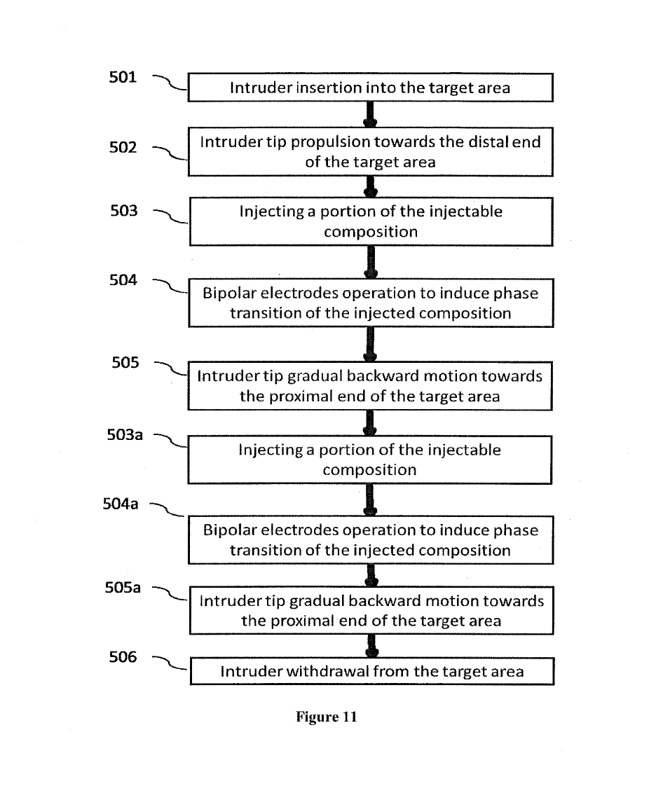

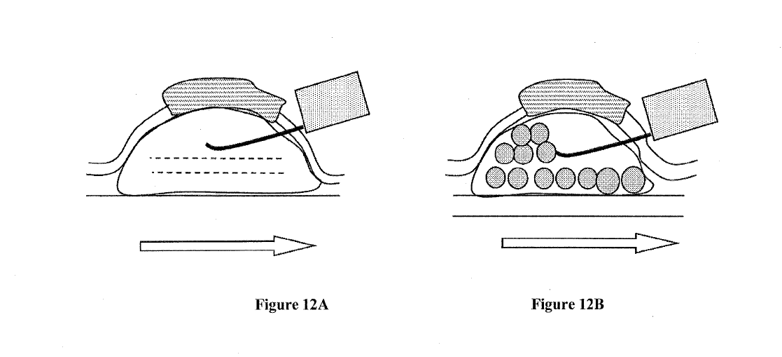

Reference is now made to FIG. 11, which schematically illustrates an operation mode of intruder 69a, described in detail in FIGS. 9A-9D, and to FIGS. 12A-12C, which schematically illustrate a tissue elevation process, wherein FIG. 12A depicts an injectable composition injection and FIG. 12B depicts phase transition of the injected composition, according to some embodiments of the invention.

The method of lifting a target tissue includes inserting the intruder into the target area (502). According to some embodiments, the user locates the optimal entry point under the target tissue while the tissue is approximately 6-12mm from the intruder distal end. After the identification of the intruder position under the target tissue by means of the endoscope's imager, the user controllably propels the intruder until the distal end of the intruder is located above the distal end of the target tissue and/or until the proximal end of the intruder contact the proximal end of the target tissue (502). While continually observing the tissue, the physician begins the injection process (503). The injection of the injectable composition is depicted in FIG. 12A. When a defined portion of the injectable composition is delivered to the target area, bipolar electrodes operation is initiated in order to induce phase transition of the injected fluid (504). The phase transition from liquid state to solid state of the injected composition is depicted in FIG. 12B. Upon solidification of the injected fluid, the intruder slides backwards, wherein the sliding is assisted by the sliding surface thereof (505). According to some embodiments, the backwards motion is performed to the distance of about 2-3 mm, while the insertion depth of the intruder tip under the target tissue is constantly controlled by the user. Upon displacement of the intruder tip, additional portion of the injectable composition is injected into the target area (503a). Said injection (503a), bipolar electrodes operation and the subsequent injected fluid phase transition (504a) and backwards sliding of the intruder (505a) are repeated until the entire target area is filled with the solidified injectable composition. Upon the desirable target tissue lifting the intruder is withdrawn from the target area (506). According to some embodiments, the method allows tissue elevation in the range from about 3 mm to about 18 mm According to the preferred embodiments, the elevation is at least about 8 mm.

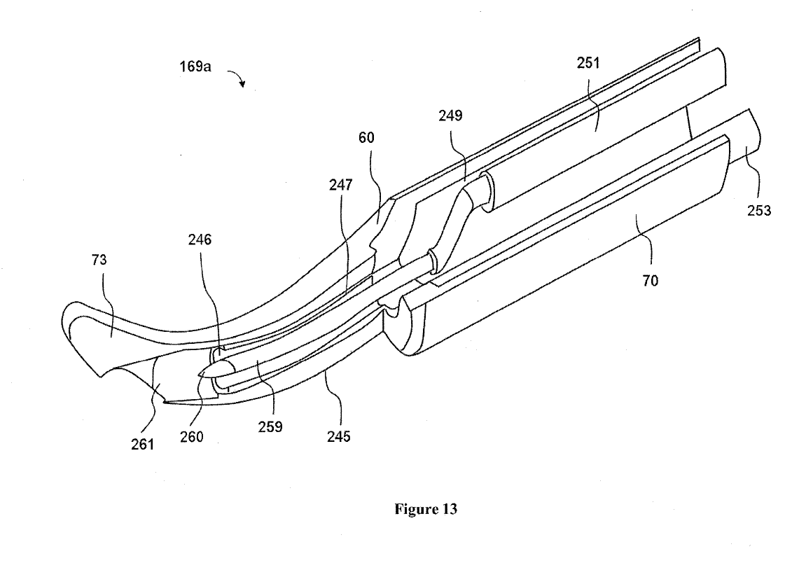

Reference is now made to FIG. 13, which schematically illustrates a cross-section view of intruder 169a, including concentric electrodes 259 and 260, according to some embodiments of the invention.

Intruder 169a includes phase transition chamber 245, including distal outlet 246 disposed at distal end 73 of intruder 169a and distal proximal inlet 247 disposed at proximal end 60 of intruder 169a. Distal outlet 247 is configured to transfer the composition in the solid state thereof to contacting delivery channel 261, wherein delivery channel 261 is configured to deliver the composition which underwent phase transition to the target area and proximal inlet 247 is configured to receive the composition in the liquid state thereof. Distal inlet 247 is connected to tube 70, wherein tube 70 is configured to transfer the composition in the liquid state thereof to phase transition chamber 245. Intruder 169a further includes central electrode 259 and hollow conical electrode 260, configured in a concentric formation, wherein central electrode 259 is disposed inside hollow conical electrode 260, while not directly contacting it. Electrodes 259 and 260 are configured to induce phase transition of the composition contained between the electrodes. Activation of the electrodes (applying the voltage) generates electric field between the electrodes and inducing current flow in the composition contained between the electrodes. The resistance of the composition to the current flow generates heat, resulting in the phase transition of the composition. According to some embodiments, electrodes 259 and 260 are bipolar. Hollow conical electrode 260 contacts the inner walls of phase transition chamber. Electrodes 259 and 260 are exposed (non-isolated) along essentially the entire length of phase transition chamber 245. The composition, entering phase transition chamber 245 from proximal outlet 249 is located between electrodes 259 and 260.Upon voltage application to the electrodes, the composition temperature increases, inducing the phase transition thereof, along essentially the entire length of exposed electrodes 259 and 260, thus providing a uniform heating and solidification of the composition. The uniform heating of the composition further prevents sticking of the solidified composition to the walls of phase transition chamber 245. The composition which underwent the phase transition is pushed from phase transition chamber 245 towards delivery channel 261 and is subsequently ejected from delivery channel 261 into the target area below intruder distal end 73. Tube 70, connected to intruder 169a further includes cables 251 and 253, connecting electrodes 259 and 260, respectively, to a power source (not shown). Tube 70, connected to intruder 69 further includes central electrode isolated connector 249, configured to screen central electrode 259 to prevent the composition phase transition inside tube 70.

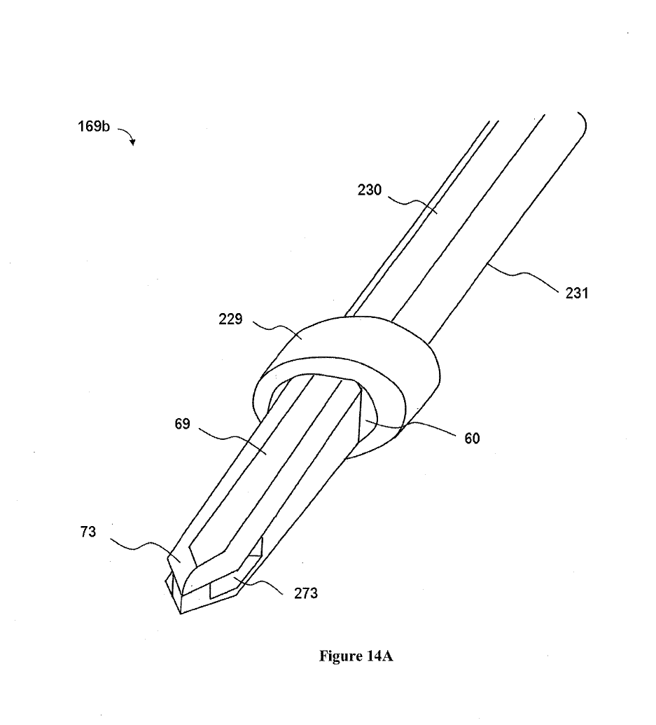

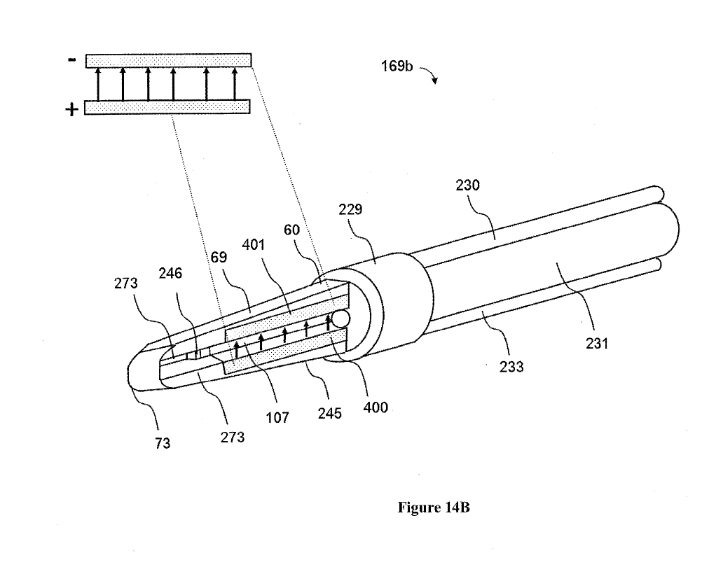

Reference is now made to FIG. 14A, which schematically illustrates intruder 169b, including parallel electrodes 400 and 401, and to FIG. 14B, which schematically illustrates a cross-sectional view of intruder 169b, according to some embodiments of the invention.

Intruder 169b includes proximal end 60 and distal end 73. Proximal end 60 is connected to tube 70 through tube adaptor 229, wherein tube 70 contains the composition configured to undergo phase transition and insulated utilities cable 230. Distal end 73 includes delivery channel 273, configured to transfer the solidified composition from phase transition chamber 245 to the target area. Phase transition chamber 245 is disposed between delivery channel 273 and intruder proximal end 60.

Intruder 169b further includes planar parallel electrodes 400 and 401, disposed inside phase transition chamber 245 and configured to induce phase transition of the composition, entering phase transition chamber 245 from tube 70 and contained between electrode 400 and electrode 401 along the entire length of the electrodes, from intruder 169b proximal end to phase transition chamber 245 distal outlet 246. Upon activation of electrodes 400 and 401 and the subsequent heating of the composition contained between the electrodes, the phase transition of the composition occurs. Solidified composition is ejected from phase transition chamber 245 distal outlet 246 and transferred to delivery channel 273. The composition, which underwent phase transition, is further transferred from delivery channel 273 to the target area. Delivery channel 273 includes a plurality of symmetrical openings, such as opening 274, configured to provide a homogeneous delivery of the composition to the target area.

According to some embodiments, the shape of top surface of intruder 169b is different from the shape of intruder 169b bottom surface, in order to allow a user to evaluate a spatial orientation of intruder 169b during the insertion and composition delivery process.

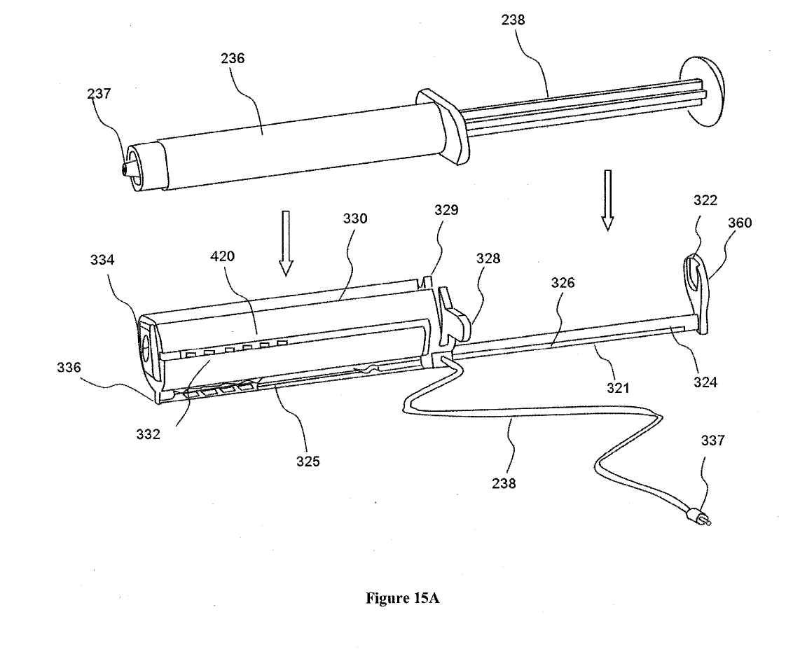

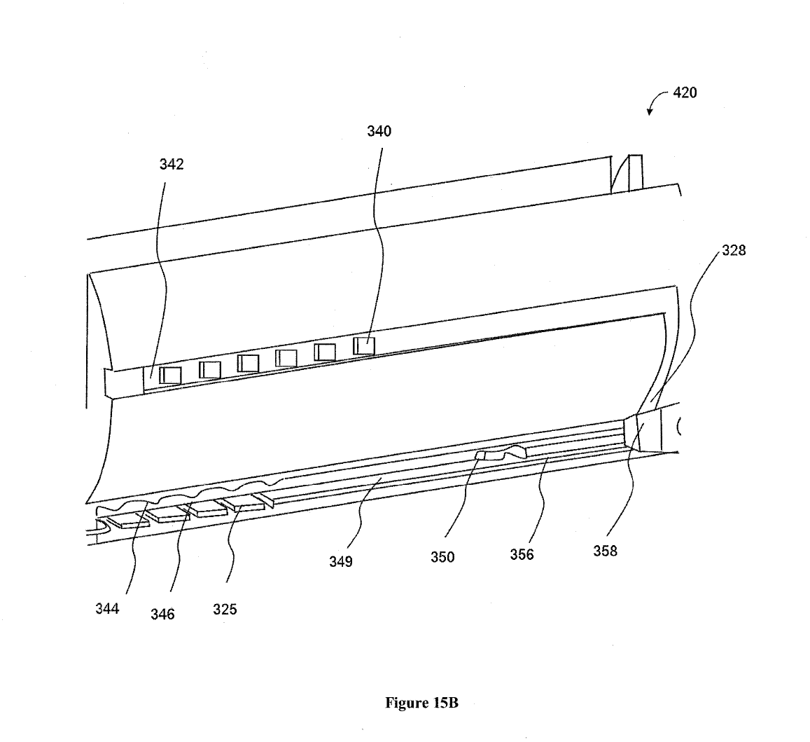

Reference is now made to FIG. 15A, which schematically illustrates dosing module 420 and storage chamber 236, according to some embodiments of the invention.

Storage chamber 236 is configured to store the composition and dosing module 420 is configured to facilitate the delivery of the composition to intruder 169a or 169b through tube 70 (not shown). Each possibility represents a separate embodiment of the invention. In the exemplary embodiment, intruder 169a and intruder 169b can be used interchangeably. Storage chamber includes opening 237, configured to provide delivery of the composition to intruder 69 and piston 238, configured to push the composition, stored inside storage chamber 236 towards opening 237.

Dosing module 420 is configured to accommodate storage chamber 236. Dosing module 420 includes two sub-assemblies: static subassembly 332, which is shaped as a cylinder with an opening and dynamic piston-pusher subassembly 321. Static subassembly 332 includes a plurality of locking clips, such as locking clip 334 on the distal side 336 thereof, further including an access to a lure connector of intruder 69 or tube 70 (not shown) inlet. Distal side 336 of static subassembly 332 is configured to connect to intruder 69 or tube 70. Static subassembly 332 further includes opening 330, configured to allow storage chamber insertion into dosing module 420. Static subassembly 332 further includes notch 329, preferably having a rectangular shape, configured to provide alignment and orientation of subassemblies 332 and 321 while in action.

Static subassembly 332 further includes ratchet mechanism 325 stretching along static subassembly 332 main axis from the proximal side to distal side 336. Static subassembly 332 further includes control wires 238 and control wires plug 337. Static subassembly 332 additionally includes holder 328, configured to allow the user gripping of static subassembly 332, while pushing dynamic subassembly 321 towards static subassembly distal side 336.

Dynamic subassembly 321 includes piston shaft 326, piston cap 360 and piston fixture 322. Piston cap 30 and piston fixture 322 are configured to immobilize piston 238 of storage chamber 236, when storage chamber 236 is inserted into dosing module 302 and piston shaft is configured to assist pushing of the piston to transfer the composition to intruder 69 through tube 70.

Reference is now made to FIG. 15B, which schematically illustrates a detailed view of dosing module 420, according to some embodiments of the invention. Ratchet mechanism 325 includes a plurality of ratchet teeth, such as ratchet tooth 344 and a plurality of station pads, such as station pad 348, contacting station pads printed circuit board (PCB) 346. Each station pads, for example, station pad 348 are configured to temporarily halt the protrusion of piston 238 (shown in FIG. 14A), by contacting ratchet tooth 344, and the plurality of station pads are, thereby, configured to control the piston motion and the amount/volume of the composition delivered to the intruder. Station pads PCB 346 is connected to control wires split box 358.

Ratchet mechanism 325 further includes a plurality of inspection LEDs, such as inspection LED 340, disposed on LED PCB 342, configured to provide indication of the position of piston 238 (shown in FIG. 15A). LED PCB 342 is connected by LED PCB cable to ratchet mechanism 325 and to control wires split box 358. LED PCB is therefore connected to station pad PCB 346, which allows providing an indication of a contact between ratchet tooth, such as ratchet tooth 344 and a station pad, such as station pad 348, and accordingly providing an indication of the piston protrusion.

Ratchet mechanism 325 further includes flex switch conductive pad 350, configured to close the control circuit when piston 238 passes a station pad, such as station pad 348 through contacting a ratchet tooth, such as ratchet tooth 344, providing an indication to activate electrodes 259 and 260 (shown in FIG. 13) of intruder 69. Activating the electrodes in response to such indication allows heating of a controlled volume of the composition, transferred to the intruder from storage chamber 236 (shown in FIG. 8B) and therefore dosed amounts of the composition, which underwent phase transition, can be delivered to the target area.

According to some embodiments, dosing module 420 can be operated by initially inserting storage chamber 236 into dosing module 420 and securing it in place. Dosing module 420 is further connected to intruder 169a or tube 70 lure port at phase transition chamber distal inlet 247 (shown in FIG. 13). Upon the injection of the composition, the dynamic piston-pusher subassembly moves along storage chamber 236, while the control wire contact strip 324 contacts control wires split box 358. The dosing module 420 operation proceeds by piston shaft 326 protrusion towards static subassembly distal side 336, wherein the protrusion of piston 326 is temporally halted, when tooth 344 contacts station pad 348 and resumed, upon additional pushing of piston 326. Each stop of piston 326 protrusion provides delivery of a predetermined amount/volume of the composition from storage chamber 236 to intruder 69. Each stop further induces electrodes 259 and 260 activation, inducing the composition phase transition. Each stop of piston 326 protrusion, therefore, facilitates delivery of predetermined amount/volume of the solidified composition to the target area. An indication of the piston protrusion stops are configured to me mechanically transmitted to the user, by sensing ratchet mechanism 325 operation. Protrusion of piston 326 and the delivery of the dosed solidified composition are performed until the piston fishes the stroke and storage chamber 236 is empty, indicating, that essentially the entire amount/volume of the composition has been delivered to intruder 69. Upon the delivery of the entire amount/volume of the composition, piston 325 can be retracted to the initial position thereof and storage chamber 236 can be replaced with a new storage chamber. Dosing module 420 operation can be repeated, until the desired amount/volume of the composition, which underwent phase transition is delivered to the target area and/or the substantial lifting of the tissue is achieved.

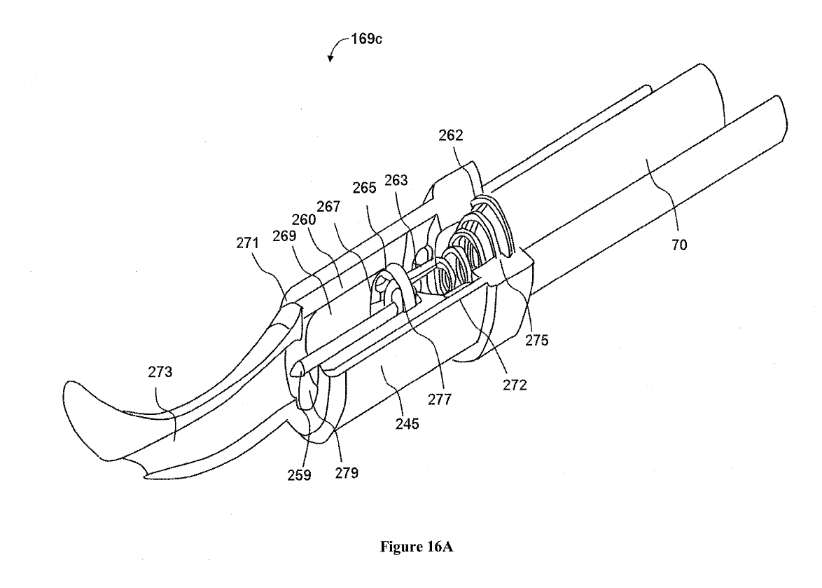

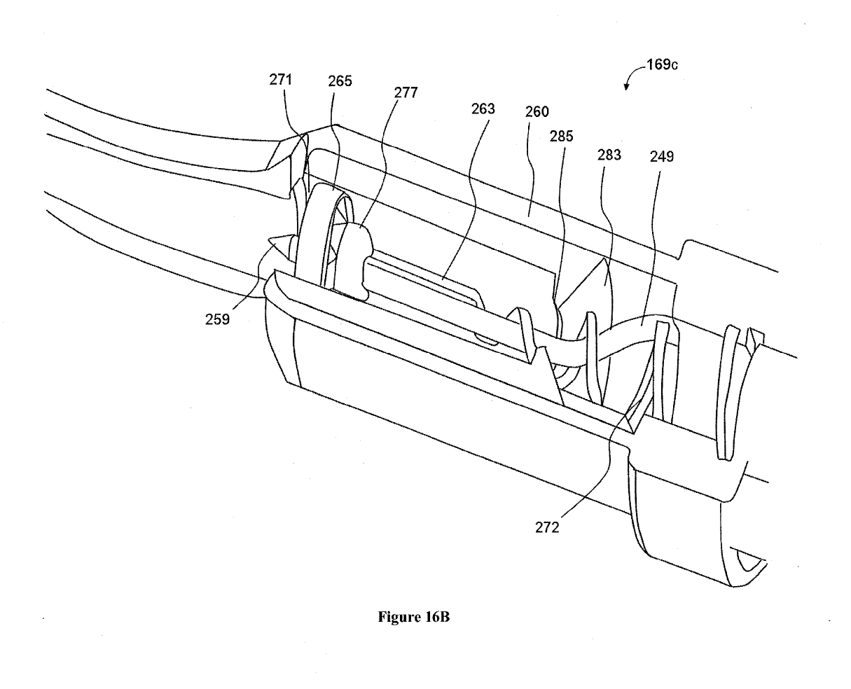

Reference is now made to FIG. 16A, which schematically illustrates actuator assisted intruder 169c, wherein piston valve 265 is in retracted position and to FIG. 16B, which schematically illustrates actuator assisted intruder 169c, wherein piston valve 265 is in extended position, according to some embodiments of the invention.

Actuator assisted intruder 169c includes phase transition chamber 245, central electrode 259 and hollow conical electrode 260, configured in a concentric formation, and is connected to tube 70, as described in FIG. 13. The inner diameter of the proximal side of phase transition chamber 245 is from about 50% to about 100% of the outer diameter of the proximal side of phase transition chamber 245. Actuator assisted intruder 169c further includes delivery channel 273, configured to transfer the composition, which underwent phase transition, from phase transition channel 245 to the target area. Actuator assisted intruder 169c additionally includes an actuator 263, configured to assist the delivery of the composition which underwent phase transition, to the target area. Actuator 263 is disposed in the proximal side of phase transition chamber 245 and the distal side of tube 70. Actuator 263 includes piston valve 265 and spring 275, associated with piston valve 265. The proximal end of spring 275 is disposed in actuator anchor slot 262 and distal side thereof contacts piston valve 265. Piston valve 265 is disposed concentrically to central electrode 259. Piston valve 259 is configured to slide along central electrode 259 between proximal stop ring 272 (as depicted in FIG. 16A) and distal stop ring 271 (as depicted in FIG. 16B). When piston valve 259 is disposed at proximal stop ring 272, spring 275 is contracted, exerting pressure on piston valve 259. Piston valve 259 contacting the composition, which underwent phase transition therefore is displaced from proximal stop ring 272, pushing the composition towards delivery channel 273 until reaching proximal stop ring 271 and/or until spring 275 is extended.

The composition in the liquid state, entering phase transition chamber 259 from tube 70, exerts additional pressure on piston valve 265, inducing the sliding thereof towards delivery channel 279. Displacement of valve piston 265 from proximal stop ring 272 allows the composition enter phase transition chamber 245.

At distal stop ring 271 there is an enlargement of the cylindrical diameter dimensions of phase transition chamber 245, which acts as a relief valve for pressurized piston valve 265, inducing the piston valve return towards proximal stop ring 272 until contacting it and preventing the additional composition entrance to phase transition chamber 245.

Once piston-valve 265 reaches distal stop ring 271, the pressure exerted by spring 275 and the liquid composition decreases, and piston valve 265 slides backwards, towards proximal stop ring 272. Piston valve 265 includes a plurality of windows, such as window 267, configured to allow the liquid composition passage through them, to be located between piston valve 265 and delivery channel 279, and therefore between electrodes 259 and 260.When piston valve 265 contacts proximal stop ring 272, the predefined amount/volume of the composition trapped inside phase transition chamber 245, ready for a phase transition process, induced by activation of electrodes 259 and 260.

When the composition, which underwent phase transition process, is ejected from phase transition chamber 245, wherein the ejection is assisted by actuator 263, the composition is pressurized by delivery channel 279 funnel shape, configured to induce the composition delivery to the target area.

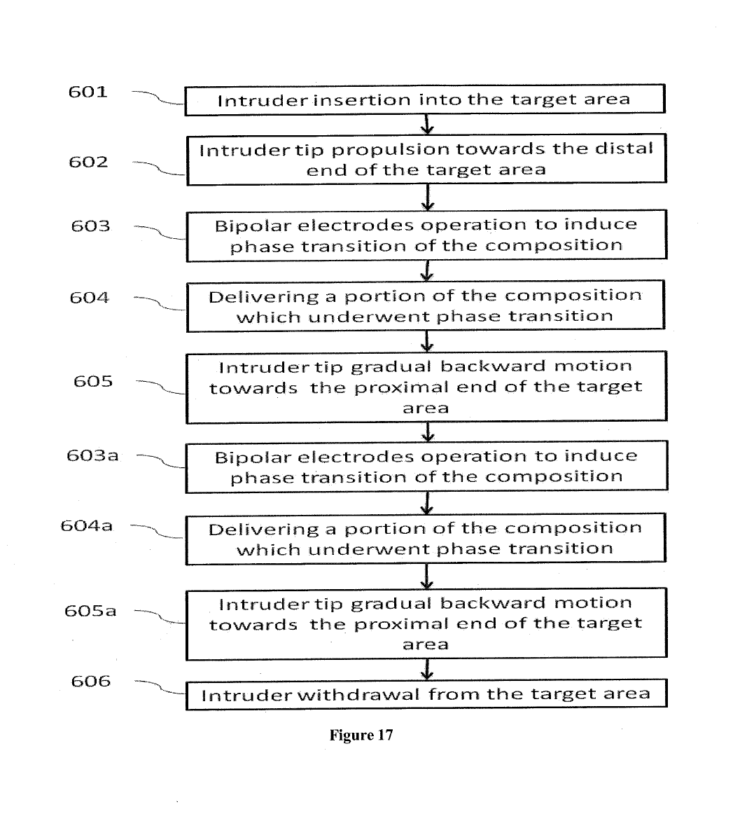

Reference is now made to FIG. 17, which schematically illustrates an operation mode of intruder 169a, described in detail in FIG. 13 and of intruder 169b, described in detail in FIG. 14A and 14B, according to some embodiments of the invention.

The method of lifting a target tissue includes inserting the intruder into the target area (602). According to some embodiments, the user locates the optimal entry point under the target tissue while the tissue is approximately 6-12mm from the intruder distal end. After the identification of the intruder position under the target tissue by means of the endoscope's imager, the user controllably propels the intruder until the distal end of the intruder is located above the distal end of the target tissue and/or until the proximal end of the intruder contacts the proximal end of the target tissue (602). While continually observing the tissue, the user activates bipolar electrodes in order to induce phase transition of the composition contained inside the intruder (603). Upon the phase transition of the composition, the composition is delivered to the target area (604). When a defined portion of the solidified composition is delivered to the target area, the intruder slides/moves backwards, wherein the sliding may be assisted by the sliding surface thereof (605). According to some embodiments, the backwards motion is performed to the distance of about 2-3 mm, while the insertion depth of the intruder tip under the target tissue is constantly controlled by the user. Upon displacement of the intruder tip, bipolar electrodes are activated and additional portion of the composition undergoes phase transition (603a). Following the phase transition, the solidified composition is delivered to the target area (604a). Said bipolar electrodes activation (and/or operation) (603a), subsequent delivery of the composition, which underwent phase transition (604a) and backwards sliding of the intruder (605a) are repeated until the entire target area is filled with the solidified composition. Upon the desirable target tissue elevation the intruder is withdrawn from the target area (606).

According to some embodiments, the method allows tissue elevation in the range from about 3 mm to about 18 mm According to the preferred embodiments, the elevation is at least about 8 mm.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope. In the description and claims of the application, each of the words “comprise” “include” and “have”, and forms thereof, are not necessarily limited to members in a list with which the words may be associated.

As used herein, the term “about”, when referring to a measurable value such as an amount, a temporal duration, and the like, is meant to encompass variations of +/−10%, more preferably +/−5%, even more preferably +/−1%, and still more preferably +/−-0.1% from the specified value, as such variations are appropriate to perform the disclosed methods.

1.-53. (canceled)

54. A method for lifting a first tissue layer with respect to a second tissue layer adjacent thereto, during an endoscopic procedure, the method comprising:
delivering a composition configured to undergo phase transition, to a region between the first tissue layer and the second tissue layer; and
controllably inducing phase transition of the composition from a liquid state to a solid state thereof.

55. The method according to claim 54, wherein the step of delivering the composition comprises delivering the composition in the liquid state thereof.

56. The method according to claim 55, wherein the step of delivering the composition precedes the step of controllably inducing the phase transition of the composition.

57. The method according to claim 54, wherein the step of delivering the composition comprises delivering the composition in the solid state thereof.

58. The method according to claim 57, wherein the step of delivering the composition follows the step of controllably inducing the phase transition of the composition.

59. The method according to claim 54, wherein the step of controllably inducing phase transition of the composition from the liquid state to the solid state comprises controlling an extent of the phase transition of the composition, a proportion of the composition which undergoes phase transition, a position of the composition which undergoes phase transition, a rate of the phase transition of the composition, a duration of the phase transition of the composition or any combination thereof.

60. The method according claim 54, wherein at least a portion of the composition being in the solid state, has a dynamic viscosity above about 60 Pa·s and/or wherein the composition in the liquid state thereof has a dynamic viscosity below about 0.15 Pa·s.

61. The method according to claim 54, wherein the step of controllably inducing phase transition of the composition from the liquid state to the solid state is performed repeatedly.

62. The method according to claim 54, wherein said controllable inducing of the phase transition of the composition provides prolonged elevation of the first tissue layer with respect to the second tissue layer for above about one hour.

63. The method according to claim 54, wherein the composition comprises a thermo-sensitive material, selected from the group consisting of proteins, hydrocolloids and combinations thereof.

64. The method according to claim 54, wherein the composition further comprises an additive, comprising a stabilizer, a color indicator, adhesion controller or a combination thereof, wherein the stabilizer is selected from the group consisting of polyoxazoline, poloxamers, polyvinylpyrrolidone (PVP) and combinations thereof and wherein the adhesion controller is selected from the group consisting of phospholipids, monoglycerides and combinations thereof.

65. A device for lifting a first tissue layer with respect to a second tissue layer adjacent thereto, during an endoscopic procedure, the device comprising:
an injection module having an elongate body, a proximal region and a distal region, wherein the distal region comprises at least one outlet, configured to deliver a composition configured to undergo phase transition, to a region between the first tissue layer and the second tissue layer; and
a phase transition module, configured to controllably induce phase transition of the composition from a liquid state to a solid state thereof.

66. The device according to claim 65, wherein the phase transition module is enclosed in the injection module and wherein the phase transition module is configured to controllably induce phase transition of the composition, disposed within the device, from the liquid state to the solid state thereof and wherein the injection module is configured to deliver the composition, in the solid state thereof.

67. The device according to claim 65, wherein the device is configured to allow controlling an extent of the phase transition of the composition, a proportion of the composition which undergoes phase transition, a position of the composition which undergoes phase transition, a rate of the phase transition of the composition, a duration of the phase transition of the composition or any combination thereof.

68. The device according to claim 66, wherein the phase transition module comprises at least one electrode, connected to a power source.

69. The device according to claim 66, wherein the phase transition module comprises concentric or planar electrodes.

70. The device according to claim 65, further comprising a cutting means disposed in the distal region of the phase transition module and/or of the injection module.

71. The device according to claim 65, wherein the distal region further comprises an orientation indicator, configured to indicate the spatial orientation of the injection module distal region and/or herein the distal region further comprises a phase transition indicator, configured to provide indication of the phase transition state of the composition.

72. The device according to claim 66, further comprising an actuator, configured to assist the delivery of the composition, in the solid state thereof, to the region between the first tissue layer and the second tissue layer.

73. The device according to claim 65, further comprising a dosing module, configured to be in a fluid-flow connection with the proximal region of the device and further configured to provide a metered delivery of the composition to the region between the first tissue layer and the second tissue layer.
